(19) United States
(12) Patent Application Publication
(10) Pub. No.: US 2020/0282021 A1
(43) Pub. Date: Sep. 10, 2020

(54) GLP-1 FUSION PROTEINS AND USES THEREOF

(71) Applicant: GENEXINE, INC., Seongnam-si (KR)

(72) Inventors: Young Chul SUNG, Seoul (KR); Mi Sun Byun, Bucheon-si (KR); In-Bok An, Seongnam-si (KR)

(73) Assignee: GENEXINE, INC., Seongnam-si (KR)

(21) Appl. No.: 16/812,687

(22) Filed: Mar. 9, 2020

Related U.S. Application Data
(60) Provisional application No. 62/815,486, filed on Mar. 8, 2019.

(51) Int. Cl. A61K 38/26; C07K 16/00; A61K 9/00; A61P 3/10

(52) U.S. Cl. CPC A61K 38/26; C07K 16/00; A61K 9/0019; A61P 3/10; A61K 2039/545; C07K 2317/524; C07K 2317/526; C07K 2317/53; C07K 2319/30

(57) ABSTRACT

A method of regulating blood glucose level and/or treating a diabetes is disclosed. The method includes administering a fusion peptide of a GLP-1 peptide and an Fc region. The Fc region is a hybrid Fc region containing a hinge region, a CH2 domain, and a CH3 domain from the N-terminal to the C-terminal direction, wherein the hinge region comprises a human IgD hinge region, the CH2 domain comprises a part of the amino acid residues of CH2 domain of human IgD and human IgG4, and the CH3 domain comprises a part of the amino acid residues of the human IgG4 CH3 domain, and has glycosylation at the IgD hinge region. The fusion peptide shows reduced side effects such as vomiting, nausea, and/or heart rate increase.

### CROSS REFERENCE TO RELATED APPLICATIONS

This application claims benefits of U.S. provisional application No. 62/815,486 filed Mar. 8, 2019, the content of which is incorporated by reference in its entirety.

### FIELD

A use of using fusion proteins of glucagon-like peptides and an Fc region in regulating blood glucose level is disclosed.

### BACKGROUND

Diabetes is associated with higher cardiovascular morbidity and mortality. Hypertension, hyperlipidemia, and diabetes are independently associated with increased risk of cardiovascular disease. Subjects with Type 2 diabetes are at two- to four-fold increased risk of cardiovascular disease compared to those without diabetes.

Glucagon-like peptide-1 (GLP-1) is known as a pleiotropic peptide with metabolic and cardiovascular benefits. It is derived from pre-proglucagon, a 158 amino acid precursor polypeptide that is processed in different tissues to form a number of different proglucagon-derived peptides. Proglucagon-derived peptides include glucagon, glucagon-like peptide-1 (GLP-1), glucagon-like peptide-2 (GLP-2), and oxyntomodulin (OXM), that are involved in a wide variety of physiological functions, including glucose homeostasis, insulin secretion, gastric emptying, and intestinal growth, as well as the regulation of food intake.

GLP-1 is produced as a 37-amino acid peptide that corresponds to amino acids 72 through 108 of proglucagon (92 to 128 of preproglucagon). The predominant biologically active form is a 30-amino acid peptide hormone (GLP-1(7-37) acid) which is produced in the gut following a meal and rapidly degraded by an abundant endogenous protease-DPP4.

Numerous GLP-1 analogs and derivatives are known. These GLP-1 analogs include the Exendins which are peptides found in the venom of the GILA-monster. The Exendins have sequence homology to native GLP-1 and can bind the GLP-1 receptor and initiate the signal transduction cascade responsible for the numerous activities that have been attributed to GLP-1(7-37)OH. These GLP-1 analogs and derivatives are referred to as “GLP-1 peptide,” GLP-1 compound,” or “GLP-1 RA” herein, and these terms are used interchangeably throughout the application.

GLP-1 peptides show a greatest promise as a treatment for non-insulin dependent diabetes mellitus. Unlike insulin of which administration can cause hypoglycemia, GLP-1 is controlled by blood glucose levels and there is no risk of hypoglycemia associated with treatment involving GLP-1 peptides.

Various long-acting GLP-1 peptides (such as GLP-1 fusion protein) with longer half-lives while maintaining multi-beneficial effects on beta cell function, insulin sensitivity, body weight and cardio-vascular system¹⁻⁴ and lack of life-threatening adverse events like hypoglycemia⁵, were intensively developed during last decades.

However, despite its attractiveness as an anti-diabetic drug, some of researches indicate that GLP-1 treatment's side effects like nausea and vomiting as well as heart rate increase could disturb the continuous growth of GLP-1 RA⁶⁻⁸.

According to cross-sectional survey by Sikirica et al., nausea/vomiting is the most contributable factors for discontinuation of GLP-1 peptides by physicians and patients with percentage of about 46% and about 64%, respectively. And about half of patients reported nausea/vomiting related factors as the most bothersome problems related to GLP-1 RAs⁸. Another potential drawback of GLP-1 peptide is heart rate increase which have been reported in the clinical trials of almost all GLP-1 peptides^{10,11}. This is likely to be the direct effect of peripherally administered GLP-1 peptides on cardiomyocytes^{12,13} which is more pronounced and sustained in long-acting GLP-1 peptides than short-acting ones⁶. Increase of heart rate by long-acting GLP-1 peptides is small but could represent safety concerns because it is one of the risk factors for the cardio-vascular disease in diabetic patients with advanced heart failure. Collectively these side effects could weaken the efficacy of GLP-1 peptides in real-world treatment thus suggesting the necessary of developing safer GLP-1 peptide to enhance therapeutic outcomes in the end.

### SUMMARY

The disclosure is directed to a use of a Fc-fused GLP-1 peptide (hereinafter, sometimes referred to as “GLP-1-gFc” or simply “fusion protein” or “fusion peptide”) with distinctive binding affinity profiles to its receptor which is designed to improve the in-vivo stability and safety. The GLP-1-gFc described herein shows good pharmacokinetic (PK) and pharmacodynamics (PD) properties as a long-acting GLP-1 RA with safer profiles compared with commercialized GLP-1 analogue such as dulaglutide.

In an aspect, a method of treating diabetes comprising administering the fusion protein to a subject in need thereof is provided. In an embodiment, the diabetes is insulin dependent. In still another embodiment, the diabetes is non-insulin dependent.

Another aspect encompasses a method of controlling or regulating glucose level in a subject comprising administering to a subject in need thereof, a fusion protein described herein.

In one embodiment, the subject has Type 2 diabetes.

In another embodiment, the subject may have metabolic syndrome.

The fusion protein comprises an IgFc and a GLP-1 peptide linked to the Fc. In one embodiment, the Fc is a hybrid comprising IgG4 CH2/CH3 moiety, IgD CH2 moiety, and IgD hinge moiety, wherein the IgD hinge moiety has glycosylation.

In another embodiment, the GLP-1 peptide can have no more than 6 amino acids that are different from the corresponding amino acid in GLP-1(7-37) (SEQ ID NO: 1), GLP-1 (7-36) (SEQ ID NO: 11), or Exendin-4 (SEQ ID NO: 10). It is even more preferred that the GLP-1 peptide have no more than 5 amino acids that differ from the corresponding amino acid in GLP-1(7-37) of SEQ ID NO: 1, GLP-1 (7-36) (SEQ ID NO: 11), or Exendin-4 of SEQ ID NO: 10. It is preferred that the GLP-1 peptide have no more than 4, 3, or 2 amino acids that differ from the corresponding amino acid in GLP-1(7-37), GLP-1(7-36) or Exendin-4. In specific embodiments, a GLP-1 peptide that is part of the fusion protein has an amino acid sequence selected from the group consisting of SEQ ID NOS: 1 and 11-34. In an embodiment, the IgD hinge moiety may have an amino acid sequence selected from the group consisting of SEQ ID NOS: 35-38.

The following exemplary modes are disclosed.
Mode 1. A method for regulating blood glucose level in a subject in need thereof, comprising administering to the subject an effective amount of a fusion peptide comprising (a) glucagon-like peptide-1 (GLP-1) peptide and (b) an immunoglobulin Fc region, wherein the immunoglobulin Fc region (b) comprises
   (i) an isolated IgD hinge region consisting of 35 to 49 consecutive amino acid residues from the C-terminus of SEQ ID NO: 3; and
   (ii) a CH2 domain and a CH3 domain of the immunoglobulin Fc polypeptide.
Mode 2. The method of mode 1, wherein the effective amount ranges from about 0.01 mg/kg to about 1 mg/kg body weight.
Mode 3. The method of any one of previous modes, wherein the fusion peptide is administered parentally at an interval of 1 week or greater.
Mode 4. The method of any one of previous modes, wherein the subject suffers from diabetes, glucose intolerance, and/or insulin resistance.
Mode 5. The method of any one of previous modes, wherein the GLP-1 peptide (a) comprises the amino acid sequence selected from the group consisting of SEQ ID NO: 1 and SEQ ID NOS: 10 to 34.
Mode 6. The method of any one of previous modes, wherein the isolated IgD hinge region (i) comprises the amino acid sequence of SEQ ID NO: 36, 37, or 38.
Mode 7. The method of any one of previous modes, wherein the immunoglobulin Fc region (b) comprises the amino acid sequence selected from the group consisting of SEQ ID NOS: 4 to 8.
Mode 8. The method of any one of previous modes, wherein the fusion peptide comprises the amino acid sequence selected from the group consisting of SEQ ID NOS: 40 to 42 and 54.
Mode 9. The method of any one of previous modes, wherein the fusion peptide is administered at a dose of 0.01 mg/kg to 0.2 mg/kg at an interval of 1 week or at a frequency of once per week.
Mode 10. The method of any one of previous modes, wherein the fusion peptide is administered at a dose of 0.2 mg/kg to 0.5 mg/kg at an interval of 2 weeks, or at a frequency of every other week.
Mode 11. The method of any one of previous modes, wherein the subject suffers from diabetes.
Mode 12. The method of any one of previous modes, wherein the diabetes is type II diabetes.
Mode 13. The method of any one of previous modes, wherein the fusion peptide is administered subcutaneously.
Mode 14. The method of any one of previous modes, wherein the fusion peptide is a dimer comprising two peptides joined together by sulfide bonds wherein the each peptide comprises the Fc region (b) of SEQ ID NO: 4, 5, 6, 7, or 8.
Mode 15. A method for preventing and/or treating diabetes in a subject in need thereof, administering to the subject an effective amount of a fusion peptide comprising (a) glucagon-like peptide-1 (GLP-1) peptide and (b) an immunoglobulin Fc region, wherein the immunoglobulin Fc region (b) comprises
   (i) an isolated IgD hinge region consisting of 35 to 49 consecutive amino acid residues from the C-terminus of SEQ ID NO: 3; and
   (ii) a CH2 domain and a CH3 domain of the immunoglobulin Fc polypeptide.
Mode 16. The method of mode 15, wherein the effective amount ranges from about 0.01 mg/kg to about 1 mg/kg body weight.
Mode 17. The method of any of modes 15-16, wherein the fusion peptide is administered parentally at an interval of 1 week or greater.
Mode 18. The method of any one of modes 15-17, wherein the fusion peptide is administered at a dose of 0.01 mg/kg to 0.2 mg/kg at an interval of 1 week or at a frequency of once per week.
Mode 19. The method of any one of modes 15-18, wherein the fusion peptide is administered at a dose of 0.2 mg/kg to 0.5 mg/kg at an interval of 2 weeks, or at a frequency of every other week.
Mode 20. The method of any one of modes 15-19, wherein the diabetes is non-insulin dependent diabetes or insulin dependent diabetes.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1(A) is a schematic diagram of dulaglutide and an embodiment of the inventive GLP-1-gFc fusion protein.

FIG. 1B) through FIG. 1(G) show that GLP-1-gFc has a higher dissociation constant (Kd) and lower receptor-mediated response. Various concentrations of GLP-1-gFc and Dulaglutide were loaded to the harvested GLP-1R expressing cells followed by 2 min treatment of Bright-Glo™ assay reagent. Luminescence was measured and plotted with concentrations of test articles (FIG. 1(B)). In-vitro activity in a transgenic cAMP-specific luciferin- and GLP-1 receptor (GLP-1R)-expressing cell line (GLP1R_cAMP/luc). Binding affinity of each test articles were evaluated by SPR (Surface plasmon resonance) analysis system (FIG. 1(C)). Sensorgram and corresponding values (Ka: association constant, Kd: dissociation constant, KD: dissociation constant at equilibrium) were presented. Results are a representative of more than two independent experiments. Result of in-vitro activity assay and SPR were presented as mean±SEM and mean of value from different concentration (FIG. 1(D)). FIG. 1(E): Pharmacokinetics of GLP-1 peptide and GLP-1-Fc after IV administration of in SD rat (n=4/group). FIG. 1(F): Pharmacokinetics of GLP-1-Fc and GLP-1-gFc after SC administration in SD rat (n=4/group). FIG. 1(G): IPGTT results of GLP-1 peptide, GLP-1-Fc, and GLP-1-gFc in CD-1 mice that were received each test molecules via SC route followed by IP challenges of 2 g/kg glucose (n=4/group/day). The AUC of changed glucose level on each day were calculated and converted to % AUC of vehicle to plot % AUC versus time. Results are presented as the mean±standard deviations for PK and mean±SEM for others. * p<0.05, ** p<0.01, ***p<0.001 vs. vehicle group, # p<0.05 vs. GLP-1-Fc group. One-way ANOVA followed by Tukey's and Dunnett's T3 test as a post-hoc analysis. T_{1/2}, half-life; AUCₗₐₛₜ, The area under the serum concentration time curve to the time of last measurable concentration.

FIG. 1(H) shows lower binding affinity of GLP-1-gFc than Dulaglutide determined by BLI system. Two graphs are representative sensorgrams of GLP-1-gFc and Dulaglutide's binding affinity, and the table shows mean of affinity parameters. The assay was repeated three times and new biosensors were used for each test article. KD, dissociation constant at equilibrium; Kₒₙ, association constant; K_{dis}, dissociation constant; R², R-squared.

FIG. 2(A) and FIG. 2(B) show the results of glucose-lowering effects of GLP-1-gFc of dulaglutide at 0.6 mg/kg body weight and GLP-1-gFc at 0.6 mg/kg body weight and at 2.4 mg/kg body weight. 6-week old male db/db mouse received weekly subcutaneous injections of indicated test articles for 6 weeks. Blood samples from tail vein were collected at weekly and biweekly for non-fasting glucose and glycated hemoglobin (HbA1c), respectively, to monitor anti-diabetic efficacy of molecules. Results were presented as mean±SEM; n=6-8/group. Statistics were assessed by student's T-test where *p<0.05, **p<0.01, ***p<0.001 vs. vehicle.

FIG. 2(C) is modeling figures of binding structure of GPL-1-Fc/GLP-1 receptor and GLP-1-gFc/GLP-1 receptor that are prepared by Pymol software. Left figure is the modeling of binding structure between GLP-1 receptor and GLP-1-gFc, and right figure is the modeling of binding structure between GLP-1 receptor and GLP-1-Fc. Structure of the GLP1-GLP1 receptor complex (PDB 3IOL) and human IgG4 (PDB 4C54) were adopted from RCSB PDB (Protein Data Bank). The Fc and gFc, which are consist of IgD and IgG4, were obtained from Phyre v2.0 software using human IgG4 Fc (PDB 4C54) as a template.

FIG. 3(A) through FIG. 3(C) show comparison of dulaglutide and GLP-1-gFc in glucose lowering and body weight in obese ob/ob mice. An equivalent dose of GLP-1-gFc and Dulaglutide were administered weekly via subcutaneous route to 9-week old female obese ob/ob mice for 4 weeks. Food intake and body weight were measured once a week during treatment period and HbA1c was measured at start and the end of the treatment period (week 0 and week 4). Results were presented as mean±SEM; n=6-8/group. Statistics were assessed by student's T-test where *p<0.05, **p<0.01, ***p<0.001 for vs. vehicle; # p<0.05 for GLP-1-gFc vs. Dulaglutide. The results show that GLP-1-gFc exhibits comparable effects on glucose lowering.

FIG. 4(A) through FIG. 4(C) show the mouse CTA and monkey ECG studies with regard to side effects (nausea and vomiting) and QT elongation responses by GLP-1-gFc and dulaglutide. To compare the CTA response by GLP-1-gFc and Dulaglutide with Positive control, LiCl, Blueberry bar consumption was measured before administration of each molecules (day 0) (a) and after 14 days of wash-out period (b). Potential effect of GLP-1-gFc and Dulaglutide on electrophysiological signals of heart was evaluated in telemetry-instrumented Cynomolgus monkeys (c). Monkeys were single administered via subcutaneous route with different doses of Dulaglutide and GLP-1-gFc. ECG waveforms were recorded from at least 2 hours before injection to approximately 24 post dose. Especially QT interval of individual monkeys was obtained and converted to QTc (corrected QT). Results were presented as mean±SEM; n=10/group for mouse CTA, n=2-3/group for monkey ECG study. Statistics were assessed by Mann-whitney test where **p<0.01, ***p<0.001 for vs. vehicle; ## p<0.01 for GLP-1-gFc vs. Dulaglutide.

FIG. 4(D) shows confirmed drug-wash out evaluated by overnight food intake before the second exposure to blueberry bar in CTA study (n=8-10/group). Overnight food intake on day 1 post-injection was dramatically reduced in the GLP-1RA-treated groups. In contrast, one day before the second exposure (day 13), overnight food intake did not differ between the GLP-1-gFc and dulaglutide groups, confirming complete wash-out of GLP-1-RA-related food intake suppression. Results are presented as means±standard errors of the mean. ***p<0.001 vs. vehicle, # p<0.01 vs. dulaglutide, Mann-Whitney U test. n.s., non-significant; Dula_0.6, dulaglutide 0.6 mg/kg; gFc_2.4, GLP-1-gFc 2.4 mg/kg.

FIG. 5(A) through FIG. 5(C) show pharmacokinetics of GLP-1-gFc (single subcutaneous administration) in healthy human subject. Six (6) escalating doses of GLP-1-gFc were administered subcutaneously in healthy men. Blood samples which were collected at indicated time points were analyzed and plotted versus post-injection time (a). The max concentration (Cₘₐₓ) and area under the curve by the last measurable time (AUCₗₐₛₜ) were plotted versus each doses to evaluate the dose-dependency of pharmacokinetics (b, c). Results were presented as mean±SD; n=6/cohort. The results show that GLP-1-gFc exhibits dose-dependent pharmacokinetics.

FIG. 5(D) shows the dose-dependent PK profiles. GLP-1-gFc shows dose-dependent PK profiles in SD rats (n=3/group) and Cynomulgus monkeys (n=3/sex/dose) after single SC administration. Collected serum samples were analyzed using GLP-1-gFc specific ELISA method where mouse Anti-human IgG4 and n-terminal specific GLP-1 antibody were used as a coating and detection antibodies. Results are presented as the mean±standard deviations. T_{1/2}, half-life.

FIG. 6(A) through FIG. 6(E) are results of evaluating side effects (nausea or vomiting, or heart rate) in oral glucose tolerance test (OGTT). Blood samples for determination of blood glucose and insulin was collected before and 0.25, 0.5, 1, 1.5, and 2 hours after intake of the 75 g glucose solution. Changes of glucose and insulin were plotted versus blood collection time points. Area under the curves of each plots were calculated and plotted versus each doses to show dose-related therapeutic effects of GLP-1-gFc (FIGS. 6(A) through 6(C)). Gastro-intestinal side effects and vital signs including pulse rate were monitored throughout the study period and at follow-up visit (day 28). Among observed gastro-intestinal side effects, nausea/vomiting were presented as number of patients who experienced each side effects at each dose group (FIG. 6(D)). Observed pulse rate data were subtracted by day 0 to show change after drug administration (FIG. 6(D). Pulse rate at day 3 and 5 were plotted versus each doses to compare with therapeutic effect in OGTT study where effects were evaluated at the same time points. Results were presented as mean±SD; n=6/cohort. The results demonstrate that GL-1-gFc show remarkably low side effects.

### DESCRIPTION

A fusion protein of the embodiments may be represented by the following chemical formula (I):

GLP-1-gFc Formula (I)

wherein,

GLP-1 is a GLP-1 peptide of SEQ ID NO: 1 or its analogs or variants, and gFc is an immunoglobulin Fc region with IgD hinge region In an embodiment, GLP-1 may have an amino acid sequence of SEQ ID NO: 1, 10, or 11, their analogs or variants wherein less than 6 amino acids of SEQ ID NO: 1, 10, or 11 are substituted.

The substitution may be performed a conservative amino acid substitution, which does not affect or gives a weak effect on the entire protein charge, i.e., polarity or hydrophobicity. For the conservative amino acid substitution, Table 1 below may be referred to.

TABLE 1

| | |
|---|---|
| Basic | Arginine (Arg, R) |
| | Lysine (Lys, K) |
| | Histidine (His, H) |
| Acidic | Glutamic acid (Glu, E) |
| | Aspartic acid (Asp, D) |
| Uncharged polar | Glutamine (Gln, O) |
| | Asparagine (Asn, N) |
| | Serine (Ser, S) |
| | Threonine (Thr, T) |
| | Tyrosine (Tyr, Y) |
| Non-polar | Phenylalanine (Phe, F) |
| | Tryptophan (Trp, W) |
| | Cystein (Cys, C) |
| | Glycine (Gly, G) |
| | Alanine (Ala, A) |
| | Valine (Val, V) |
| | Proline (Pro, P) |
| | Methionine (Met, M) |
| | Leucine (Leu, L) |
| | Norleucine |
| | Isoleucine |

For each amino acid, additional conservative substitution includes “a homolog” of the amino acid. In particular, the “homolog” refers to an amino acid, in which a methylene group (CH₂) is inserted to the side chain of the beta position of the side chain of the amino acid. Examples of the “homolog” may include homophenylalanine, homoarginine, homoserine, etc., but is not limited thereto.

In an embodiment, gFc of the Formula (I) is an Fc region of a modified immunoglobulin or a part thereof, or a variant thereof, which has an IgD hinge region. The IgD hinge region has an O-glycan.

In particular, the Fc region of the modified immunoglobulin may be one in which the antibody-dependent cellular cytotoxicity (ADCC) or complement-dependent cytotoxicity (CDC) weakened due to the modification in the binding affinity with the Fc receptor and/or a complement. The modified immunoglobulin may be selected from the group consisting of IgG1, IgG2, IgG3, IgG4, IgA1, IgA2, IgD, IgE and a combination thereof. Specifically, the Fc region of the modified immunoglobulin may include a hinge region, a CH2 domain, and a CH3 domain from the N-terminal to the C-terminal. In particular, the hinge region may include the human IgD hinge region; the CH2 domain may include a part of the amino acid residues of the human IgD and a part of the amino acid residues of the human IgG4 CH2 domain; and the CH3 domain may include a part of the amino acid residues of the human IgG4 CH3 domain.

Additionally, two fusion proteins may form a dimer. For example, the Fc regions may bind to each other and thereby form a dimer. As used herein, the terms “Fc region”, “Fc fragment”, or “Fc” refers to a protein which includes the heavy chain constant region 2 (CH2) and the heavy chain constant region 3 (CH3) of immunoglobulin but does not include its variable regions of the heavy chain and the light chain and the light chain constant region (CL1), and it may further include a hinge region of the heavy chain constant region.

In an embodiment, a hybrid Fc or a hybrid Fc fragment thereof may be called “hFc” or “hyFc.”

Additionally, as used herein, the term “an Fc region variant” refers to one which was prepared by substituting a part of the amino acids among the Fc region or by combining the Fc regions of different kinds. The Fc region variant can prevent from being cut off at the hinge region. Specifically, the 144^{th} amino acid and/or 145^{th} amino acid of SEQ ID NO: 4 may be modified. Preferably, the variant may be one, in which the 144^{th} amino acid, K, was substituted with G or S, and one, in which the 145^{th} amino acid, E, was substituted with G or S. The Fc region or the Fc region variant of the modified immunoglobulin may be represented by the following Formula (II):

N′—(Z1)p-Y—Z2-Z3-Z4-C′ Formula (II).

In the above Formula (II),

N′ is the N-terminal of a polypeptide and C′ is the C-terminal of a polypeptide;

p is an integer of 0 or 1;

Z1 is an amino acid sequence having 5 to 9 consecutive amino acid residues from the amino acid residue at position 98 toward the N-terminal, among the amino acid residues at positions from 90 to 98 of SEQ ID NO: 2;

Y is an amino acid sequence having 5 to 64 consecutive amino acid residues from the amino acid residue at position 162 toward the N-terminal, among the amino acid residues at positions from 99 to 162 of SEQ ID NO: 2;

Z2 is an amino acid sequence having 4 to 37 consecutive amino acid residues from the amino acid residue at position 163 toward the C-terminal, among the amino acid residues at positions from 163 to 199 of SEQ ID NO: 2;

Z3 is an amino acid sequence having 71 to 106 consecutive amino acid residues from the amino acid residue at position 220 toward the N-terminal, among the amino acid residues at positions from 115 to 220 of SEQ ID NO:3; and

Z4 is an amino acid sequence having 80 to 107 consecutive amino acid residues from the amino acid residue at position 221 toward the C-terminal, among the amino acid residues at positions from 221 to 327 of SEQ ID NO: 3.

Additionally, the Fc fragment may be in the form of having native sugar chains, increased sugar chains, or decreased sugar chains compared to the native form. The immunoglobulin Fc sugar chains may be modified by conventional methods such as a chemical method, an enzymatic method, and a genetic engineering method using a microorganism.

Additionally, the Fc region of the modified immunoglobulin may include the amino acid sequence of SEQ ID NO: 4 (hyFc), SEQ ID NO: 5 (hyFcM1), SEQ ID NO: 6 (hyFcM2), SEQ ID NO: 7 (hyFcM3), or SEQ ID NO: 8 (hyFcM4). Additionally, the Fc region of the modified immunoglobulin may include the amino acid sequence of SEQ ID NO: 9 (a non-lytic mouse Fc). The Fc region of the modified immunoglobulin may be one described in U.S. Pat. No. 7,867,491, and the production of the Fc region of the modified immunoglobulin may be performed referring to the disclosure in U.S. Pat. No. 7,867,491, the entire content of which is incorporated herein by reference. The gFc of Formula (I) can be an immunoglobulin region comprising (i) an isolated IgD hinge region consisting of 35 to 49 consecutive amino acid residues from the C-terminus of SEQ ID NO: 35; and (ii) a CH2 domain and a CH3 domain of the immunoglobulin Fc polypeptide. In an embodiment, the IgD hinge region comprises the amino acid sequence selected from the group consisting of SEQ ID NOs: 36-38.

The fusion protein of Formula (I) may be one described in U.S. Pat. No. 10,538,569, of which entire disclosure is incorporated herein by reference. The GLP-1 of Formula (I) may comprise the amino acid sequence selected from the group consisting of SEQ ID NO: 1 and SEQ ID NO: 11-34.

A nucleic acid construct (or a genomic construct) including a nucleic acid encoding the fusion protein may be used as a part of the gene therapy protocol. For reconstituting or complementing the functions of a desired protein, an expression vector capable of expressing a fusion protein in a particular cell may be administered along with any biologically effective carrier. This may be any formulation or composition that can efficiently deliver a gene encoding the fusion protein into a cell in vivo.

The GLP-1 and the gFc may be fused through a peptide linker. The peptide linker may be a peptide of 10 to 20 amino acid residues consisting of Gly and Ser residues.

In an embodiment, the C-terminal of GLP-1 peptide may be fused to the N-terminus of the Fc region.

In an embodiment, the fusion protein of formula (I) has an amino acid sequence of SEQ ID NOs: 40, 41, 42, or 54.

The fusion protein may be produced by expressing in a nucleic acid encoding the fusion protein in a proper host.

The nucleic acid molecule may further include a signal sequence or a leader sequence.

As used herein, the term “signal sequence” refers to a fragment directing the secretion of a biologically active molecule drug and a fusion protein, and it is cut off after being translated in a host cell. The signal sequence of an embodiment is a polynucleotide encoding an amino acid sequence initiating the movement of the protein penetrating the endoplasmic reticulum (ER) membrane. The useful signal sequences in an embodiment include an antibody light chain signal sequence, e.g., antibody 14.18 (Gillies et al., J. Immunol. Meth 1989. 125:191-202), an antibody heavy chain signal sequence, e.g., MOPC141 an antibody heavy chain signal sequence (Sakano et al., Nature, 1980. 286: 676-683), and other signal sequences know in the art (e.g., see Watson et al., Nucleic Acid Research, 1984. 12:5145-5164).

The characteristics of the signal peptides are well known in the art, and the signal peptides conventionally having 16 to 30 amino acids, but they may include more or less number of amino acid residues. Conventional signal peptides consist of three regions of the basic N-terminal region, a central hydrophobic region, and a more polar C-terminal region.

The central hydrophobic region includes 4 to 12 hydrophobic residues, which immobilize the signal sequence through a membrane lipid bilayer during the translocation of an immature polypeptide. After the initiation, the signal sequence is frequently cut off within the lumen of ER by a cellular enzyme known as a signal peptidase. In particular, the signal sequence may be a secretory signal sequence for tissue plasminogen activation (tPa), signal sequence of herpes simplex virus glycoprotein D (HSV gDs), or a growth hormone. Preferably, the secretory signal sequence used in higher eukaryotic cells including mammals, etc., may be used. Additionally, as the secretory signal sequence, the signal sequence included in the GLP-1 may be used or it may be used after substituting with a codon with high expression frequency in a host cell.

An isolated nucleic acid molecule encoding the fusion protein may be contained in an expression vector.

As used herein, the term “vector” is understood as a nucleic acid means which includes a nucleotide sequence that can be introduced into a host cell to be recombined and inserted into the genome of the host cell, or spontaneously replicated as an episome. The vector may include linear nucleic acids, plasmids, phagemids, cosmids, RNA vectors, virus vectors, and analogs thereof. Examples of the virus vectors may include retroviruses, adenoviruses, and adeno-associated viruses, but are not limited thereto.

As used herein, the term “gene expression” or “expression” of a target protein is understood to refer to transcription of a DNA sequence, translation of an mRNA transcript, and secretion of a fusion protein product or a fragment thereof.

As used herein, the term “gene expression” or “expression” of a target protein is understood to refer to transcription of a DNA sequence, translation of an mRNA transcript, and secretion of an Fc fusion protein product or an antibody or an antibody fragment thereof.

The useful expression vector may be RcCMV (Invitrogen, Carlsbad) or a variant thereof. The expression vector may include a human cytomegalovirus (CMV) for promoting continuous transcription of a target gene in a mammalian cell and a polyadenylation signal sequence of bovine growth hormone for increasing the stability state of RNA after transcription. In an exemplary embodiment, the expression vector is pAD15, which is a modified form of RcCMV.

The expression vector may be included in an appropriate host cell suitable for the expression and/or secretion of a target protein, by the transduction or transfection of the DNA sequence of an embodiment.

As used herein, the term “host cell” or “host” refers to a prokaryotic cell and a eukaryotic cell to which a recombinant expression vector can be introduced. As used herein, the terms “transduced”, “transformed”, and “transfected” refer to the introduction of a nucleic acid (e.g., a vector) into a cell using a technology known in the art.

Examples of the appropriate host cell may include immortal hybridoma cell, NS/0 myeloma cell, 293 cell, Chinese hamster ovary (CHO) cell, HeLa cell, human amniotic fluid-derived cell (CapT cell), TM4, W138, Hep G2, MMT 060562, or COS cell. Examples of invertebrate cells include insect cells such as Drosophila S2 and Spodoptera Sp, Spodoptera high5 as well as plant cells

Nucleic acid molecule encoding the GLP-1 peptide can be made by a known method including cloning methods like those described above as well as chemically synthesized DNA. Chemical synthesis can be used given the short length of the encoded peptide. The amino acid sequence for GLP-1 has been published as well as the sequence of the preproglucagon gene. [Lopez, et al. (1983) Proc. Natl. Acad. Sci., USA 80:5485-5489; Bell, et al. (1983) Nature, 302:716-718; Heinrich, G., et al. (1984) Endocrinol, 115:2176-2181; Ghiglione, M., et al. 91984) Diabetologia 27:599-600]. Thus, primers can be designed based on the native sequence to generate DNA encoding the GLP-1 peptides.

The gene encoding a fusion protein can then be constructed by ligating a nucleic acid encoding a GLP-1 peptide in-frame to a nucleic acid encoding the Fc region described herein. A DNA encoding wild-type GLP-1 and IgG4 Fc fragments can be mutated either before ligation or in the context of a cDNA encoding an entire fusion protein, by employing known mutagenesis techniques. The gene encoding the GLP-1 peptide and the gene encoding the Fc region (e.g., gene encoding the hyFc of SEQ ID NO: 4) can also be joined in-frame directly or via DNA encoding a G-rich linker peptide.

Various forms of a fusion protein may be recovered from culture medium or from host cell lysates. If membrane-bound, it can be released from the membrane using a suitable detergent solution (e.g., Triton-X 100) or by enzymatic cleavage. Cells employed in expression of a fusion protein can be disrupted by various physical or chemical means, such as freeze-thaw cycling, sonication, mechanical disruption, or cell lysing agents.

Once the fusion proteins are expressed in the appropriate host cell, the fusion proteins can be isolated and purified. The following procedures are exemplary of suitable purification procedures: fractionation on carboxymethyl cellulose; gel filtration such as Sephadex G-75; anion exchange resin such as DEAE or Mono-Q; cation exchange such as CM or Mono-S; metal chelating columns to bind epitope-tagged forms of the polypeptide; reversed-phase HPLC; chromatofocusing; silica gel; ethanol precipitation; and ammonium sulfate precipitation.

Various methods of protein purification may be employed and such methods are known in the art and described, for example, in Deutscher, Methods in Enzymology 182: 83-9 (1990) and Scopes, Protein Purification: Principles and Practice, Springer-Verlag, NY (1982). The purification step(s) selected will depend on the nature of the production process used and the particular fusion protein produced. For example, fusion proteins comprising an Fc fragment can be effectively purified using a Protein A or Protein G affinity matrix. Low or high pH buffers can be used to elute the fusion protein from the affinity matrix. Mild elution conditions will aid in preventing irreversible denaturation of the fusion protein.

The fusion proteins may be formulated with one or more pharmaceutically acceptable carrier or excipients. The fusion proteins may be combined with a pharmaceutically acceptable buffer, and the pH adjusted to provide acceptable stability, and a pH acceptable for administration such as parenteral administration. Optionally, one or more pharmaceutically acceptable anti-microbial agents may be added. Meta-cresol and phenol are preferred pharmaceutically acceptable microbial agents. One or more pharmaceutically acceptable salts may be added to adjust the ionic strength or tonicity. One or more excipients may be added to further adjust the isotonicity of the formulation. Glycerin is an example of an isotonicity-adjusting excipient. Pharmaceutically acceptable means suitable for administration to a human or other animal and thus, does not contain toxic elements or undesirable contaminants and does not interfere with the activity of the active compounds therein.

The fusion proteins may be formulated as a solution formulation or as a lyophilized powder that can be reconstituted with an appropriate diluent. A lyophilized dosage form is one in which the fusion protein is stable, with or without buffering capacity to maintain the pH of the solution over the intended in-use shelf-life of the reconstituted product. It is preferable that the solution comprising the heterologous fusion proteins discussed herein before lyphilization be substantially isotonic to enable formation of isotonic solutions after reconstitution.

A pharmaceutically-acceptable salt form of the fusion proteins are also within the scope of the invention. Acids commonly employed to form acid addition salts are inorganic acids such as hydrochloric acid, hydrobromic acid, hydriodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Preferred acid addition salts are those formed with mineral acids such as hydrochloric acid and hydrobromic acid.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

The fusion proteins of the present invention have biological activity. Biological activity refers to the ability of the fusion protein to bind to and activate the GLP-1 receptor in vivo and elicit a response. Responses include, but are not limited to, secretion of insulin, suppression of glucagon, inhibition of appetite, weight loss, induction of satiety, inhibition of apoptosis, induction of pancreatic beta cell proliferation, and differentiation of pancreatic beta cells. A representative number of GLP-1 fusion proteins were tested for in vitro as well as in vivo activity.

Administration of the fusion proteins may be via any route known to be effective by the physician of ordinary skill. Peripheral parenteral is one such method. Parenteral administration is commonly understood in the medical literature as the injection of a dosage form into the body by a sterile syringe or some other mechanical device such as an infusion pump. Peripheral parenteral routes can include intravenous, intramuscular, subcutaneous, and intraperitoneal routes of administration.

The fusion proteins may also be administered by oral, rectal, nasal, or lower respiratory routes.

The fusion proteins can be used to regulate or normalize the blood glucose in vivo. The fusion proteins primarily exert their biological effects by acting as a GLP-1 receptor agonist, i.e., binding at a receptor referred to as the GLP-1 receptor. Subjects with diseases and/or conditions that respond favorably to GLP-1 receptor stimulation or to the administration of GLP-1 compounds can therefore be treated with the GLP-1 fusion proteins.

These subjects are said to “be in need of treatment with GLP-1 compounds” or “in need of GLP-1 receptor stimulation.” Such subject may include those with non-insulin dependent diabetes, insulin dependent diabetes, stroke (see WO 00/16797), myocardial infarction (see WO 98/08531), obesity (see WO 98/19698), catabolic changes after surgery (see U.S. Pat. No. 6,006,753), functional dyspepsia and irritable bowel syndrome (see WO 99/64060). Also included are subjects requiring prophylactic treatment with a GLP-1 compound, e.g., subjects at risk for developing non-insulin dependent diabetes (see WO 00/07617). Subjects with impaired glucose tolerance or impaired fasting glucose, subjects whose body weight is about 25% above normal body weight for the subject's height and body build, subjects with a partial pancreatectomy, subjects having one or more parents with non-insulin dependent diabetes, subjects who have had gestational diabetes and subjects who have had acute or chronic pancreatitis are at risk for developing non-insulin dependent diabetes.

An effective amount of the GLP-1-gFc fusion proteins is the quantity which results in a desired therapeutic and/or prophylactic effect without causing unacceptable side-effects when administered to a subject in need of GLP-1 receptor stimulation. A “desired therapeutic effect” includes one or more of the following: 1) an amelioration of the symptom(s) associated with the disease or condition; 2) a delay in the onset of symptoms associated with the disease or condition; 3) increased longevity compared with the absence of the treatment; and 4) greater quality of life compared with the absence of the treatment. For example, an “effective amount” of a GLP-1-gFc fusion protein for the treatment of diabetes is the quantity that would result in greater control of blood glucose concentration than in the absence of treatment, thereby resulting in a delay in the onset of diabetic complications such as retinopathy, neuropathy or kidney disease. An “effective amount” of a GLP-1-gFc fusion protein for the prevention of diabetes is the quantity that would delay, compared with the absence of treatment, the onset of elevated blood glucose levels that require treatment with anti-hypoglycaemic drugs such as sulfonyl ureas, thiazolidinediones, insulin and/or bisguanidines.

The GLP-1-gFc fusion proteins disclosed herein show lower incidents of side effects such as vomiting, nausea, and/or heart rate increase, compared to commercially available GLP-1 fusion protein drug such as dulaglutide.

The dose of fusion protein effective to normalize a patient's blood glucose will depend on a number of factors, among which are included, without limitation, the subject's sex, weight and age, the severity of inability to regulate blood glucose, the route of administration and bioavailability, the pharmacokinetic profile of the fusion protein, the potency, and the formulation. Doses may be in the range of 0.01 to 10 mg/kg body weight. In an embodiment, the doses may be in the range of 0.05 to 5 mg/kg body weight. In another embodiment, the doses may be in the range of 0.01 to 1 mg/kg body weight. In still another embodiment, the doses may be in the range of 0.05 to 0.5 mg/kg body weight. In still another embodiment, the doses may be in the range of 0.05 to 1 mg/kg body weight.

The fusion proteins can be administered at an interval of one week or greater. Depending on the disease being treated, it may be necessary to administer the fusion protein more frequently than the one week interval, such as two to three time per week.

For example, in accordance with embodiments, the doses may be administered at an interval of 1 week or greater. In one embodiment, the doses may be administered at an interval of 2 weeks or greater. In another embodiment, the doses may be administered at an interval of 3 weeks or greater. In still another embodiment, the doses may be administered at an interval of 3 days, 4 days, 5 days, 6 days, 7 days, 8 days, 9 days, 10 days, 15 days, 20 days, 30 days, 40 days, or greater. In another embodiment, the doses may be administered at a frequency of once a week, twice a week, once every other week, or twice per month, three times per month, and the like.

In an aspect, a method for lowering glucose level in a subject without or with reduced side effects, wherein the fusion protein is administered, is provided. In an embodiment, the side effect is one or more of nausea, vomiting, increased heart rate. In an embodiment, the subject has a diabetes. In an aspect, the subject has a type II diabetes.

Accordingly, in an aspect, a method for treating diabetes of a subject by administering the fusion protein is provided.

In an aspect, the method comprises administering the fusion protein at a dose of from about 0.01 mg/kg to about 10 mg/kg, about 0.02 mg/kg to about 10 mg/kg, from about 0.03 mg/kg to about 10 mg/kg, about 0.04 mg/kg to about 10 mg/kg, from about 0.05 mg/kg to about 10 mg/kg, about 0.06 mg/kg to about 10 mg/kg, from about 0.07 mg/kg to about 10 mg/kg, from about 0.08 mg/kg to about 10 mg/kg, from about 0.09 mg/kg to about 10 mg/kg, from about 0.1 mg/kg to about 10 mg/kg, from about 0.15 mg/kg to about 10 mg/kg, from about 0.2 mg/kg to about 10 mg/kg, from about 0.25 mg/kg to about 10 mg/kg, from about 0.3 mg/kg to about 10 mg/kg, from about 0.35 mg/kg to about 10 mg/kg, from about 0.4 mg/kg to about 10 mg/kg, from about 0.45 mg/kg to about 10 mg/kg, from about 0.5 mg/kg to about 10 mg/kg, from about 0.55 mg/kg to about 10 mg/kg, from about 0.6 mg/kg to about 10 mg/kg, from about 0.65 mg/kg to about 10 mg/kg, from about 0.7 mg/kg to about 10 mg/kg, from about 0.75 mg/kg to about 10 mg/kg, from about 0.8 mg/kg to about 10 mg/kg, from about 0.85 mg/kg to about 10 mg/kg, from about 0.9 mg/kg to about 10 mg/kg, from about 0.95 mg/kg to about 10 mg/kg, from about 1 mg/kg to about 10 mg/kg, from about 1.1 mg/kg to about 10 mg/kg, from about 1.2 mg/kg to about 10 mg/kg, from about 1.3 mg/kg to about 10 mg/kg, from about 1.4 mg/kg to about 10 mg/kg, from about 1.5 mg/kg to about 10 mg/kg, from about 1.6 mg/kg to about 10 mg/kg, from about 1.7 mg/kg to about 10 mg/kg, from about 1.8 mg/kg to about 10 mg/kg, from about 1.9 mg/kg to about 10 mg/kg, from about 2 mg/kg to about 10 mg/kg, from about 2.1 mg/kg to about 10 mg/kg, from about 2.2 mg/kg to about 10 mg/kg, from about 2.3 mg/kg to about 10 mg/kg, from about 2.4 mg/kg to about 10 mg/kg, from about 2.5 mg/kg to about 10 mg/kg, from about 2.6 mg/kg to about 1.0 mg/kg, from about 2.7 mg/kg to about 10 mg/kg, from about 2.8 mg/kg to about 10 mg/kg, from about 2.9 mg/kg to about 10 mg/kg, from about 3 mg/kg to about 10 mg/kg, from about 3.1 mg/kg to about 10 mg/kg, from about 3.2 mg/kg to about 10 mg/kg, from about 3.3 mg/kg to about 10 mg/kg, from about 3.4 mg/kg to about 10 mg/kg, from about 3.5 mg/kg to about 10 mg/kg, from about 3.6 mg/kg to about 10 mg/kg, from about 3.7 mg/kg to about 10 mg/kg, from about 3.8 mg/kg to about 10 mg/kg, from about 3.9 mg/kg to about 10 mg/kg, or from about 4 mg/kg to about 10 mg/kg, at an interval of 1 week or greater, at an interval of 2 weeks or greater, at an interval of 3 weeks or greater, or at an interval of 4 weeks or greater. In embodiments, the upper limit of the above ranges may be about 5 mg/kg. In another embodiments, the doses may be administered at an interval of 3 days, 4 days, 5 days, 6 days, 7 days, 8 days, 9 days, 10 days, 15 days, 20 days, 30 days, 40 days, or greater. In another embodiment, the doses may be administered at a frequency of once a week, twice a week, once every other week, or twice per month, three times per month, and the like.

In another embodiment, the fusion protein may be administered at a dose of from about 0.01 mg/kg to about 1 mg/kg, about 0.02 mg/kg to about 1 mg/kg, from about 0.03 mg/kg to about 1 mg/kg, about 0.04 mg/kg to about 1 mg/kg, from about 0.05 mg/kg to about 1 mg/kg, about 0.06 mg/kg to about 1 mg/kg, from about 0.07 mg/kg to about 1 mg/kg, from about 0.08 mg/kg to about 1 mg/kg, from about 0.09 mg/kg to about 1 mg/kg, from about 0.1 mg/kg to about 1 mg/kg, from about 0.16 mg/kg to about 1 mg/kg, from about 0.2 mg/kg to about 1 mg/kg, from about 0.24 mg/kg to about 1 mg/kg, from about 0.3 mg/kg to about 1 mg/kg, from about 0.35 mg/kg to about 1 mg/kg, from about 0.4 mg/kg to about 1 mg/kg, from about 0.45 mg/kg to about 1 mg/kg, from about 0.5 mg/kg to about 1 mg/kg, from about 0.55 mg/kg to about 1 mg/kg, from about 0.6 mg/kg to about 1 mg/kg, from about 0.65 mg/kg to about 1 mg/kg, from about 0.7 mg/kg to about 1 mg/kg, from about 0.75 mg/kg to about 1 mg/kg, from about 0.8 mg/kg to about 1 mg/kg, from about 0.85 mg/kg to about 1 mg/kg, from about 0.9 mg/kg to about 1 mg/kg, from about 0.95 mg/kg to about 1 mg/kg, at an interval of 1 week or greater, at an interval of 2 weeks or greater, at an interval of 3 weeks or greater, or at an interval of 4 weeks or greater. In still another embodiment, the doses may be administered at an interval of 3 days, 4 days, 5 days, 6 days, 7 days, 8 days, 9 days, 10 days, 15 days, 20 days, 30 days, 40 days, or greater. In another embodiment, the doses may be administered at a frequency of once a week, twice a week, once every other week, or twice per month, three times per month, and the like.

In another aspect, the method comprises administering the fusion protein at a dose of from about 0.1 mg/kg to about 5 mg/kg, from about 0.2 mg/kg to about 5 mg/kg, from about 0.3 mg/kg to about 5 mg/kg, from about 0.4 mg/kg to about 5 mg/kg, from about 0.5 mg/kg to about 5 mg/kg, from about 0.6 mg/kg to about 5 mg/kg, from about 0.7 mg/kg to about 5 mg/kg, from about 0.8 mg/kg to about 5 mg/kg, from about 0.9 mg/kg to about 5 mg/kg, from about 1 mg/kg to about 5 mg/kg, from about 1.1 mg/kg to about 5 mg/kg, from about 1.2 mg/kg to about 5 mg/kg, from about 1.3 mg/kg to about 5 mg/kg, from about 1.4 mg/kg to about 5 mg/kg, from about 1.5 mg/kg to about 5 mg/kg, from about 1.6 mg/kg to about 5 mg/kg, from about 1.7 mg/kg to about 5 mg/kg, from about 1.8 mg/kg to about 5 mg/kg, from about 1.9 mg/kg to about 5 mg/kg, from about 2 mg/kg to about 5 mg/kg, from about 2.1 mg/kg to about 5 mg/kg, from about 2.2 mg/kg to about 5 mg/kg, from about 2.3 mg/kg to about 5 mg/kg, from about 2.4 mg/kg to about 5 mg/kg, from about 2.5 mg/kg to about 5 mg/kg, from about 2.6 mg/kg to about 5 mg/kg, from about 2.7 mg/kg to about 5 mg/kg, from about 2.8 mg/kg to about 5 mg/kg, from about 2.9 mg/kg to about 5 mg/kg, from about 3 mg/kg to about 5 mg/kg, from about 3.1 mg/kg to about 5 mg/kg, from about 3.2 mg/kg to about 5 mg/kg, from about 3.3 mg/kg to about 5 mg/kg, from about 3.4 mg/kg to about 5 mg/kg, from about 3.5 mg/kg to about 5 mg/kg, from about 3.6 mg/kg to about 5 mg/kg, from about 3.7 mg/kg to about 5 mg/kg, from about 3.8 mg/kg to about 5 mg/kg, from about 3.9 mg/kg to about 5 mg/kg, or from about 4 mg/kg to about 5 mg/kg, at an interval of 3 days, 4 days, 5 days, 6 days, 7 days, 8 days, 9 days, 10 days, 15 days, 20 days, 30 days, 40 days, or greater. In another embodiment, the doses may be administered at a frequency of once a week, twice a week, once every two weeks, once per month, twice per month, three times per month, and the like.

In an exemplary embodiment, the fusion protein is administered at a dose of 0.05 mg/kg, 0.06 mg/kg, 0.07 mg/kg, 0.08 mg/kg, 0.09 mg/kg, 0.1 mg/kg, 0.11 mg/kg, 0.12 mg/kg, 0.13 mg/kg, 0.14 mg/kg, 0.15 mg/kg, 0.16 mg/kg, 0.17 mg/kg, 0.18 mg/kg, 0.19 mg/kg, 0.2 mg/kg, 0.21 mg/kg, 0.22 mg/kg, 0.23 mg/kg, 0.24 mg/kg, 0.25 mg/kg, 0.26 mg/kg, 0.27 mg/kg, 0.28 mg/kg, 0.29 mg/kg, or 3 mg/kg at an interval of 1 week or two weeks. It should be understood that the two weeks interval schedule may be replaced with a frequency of every other week.

In another exemplary embodiment, the fusion protein is administered at a dose of 0.05 mg/kg, 0.06 mg/kg, 0.07 mg/kg, 0.08 mg/kg, 0.09 mg/kg, 0.1 mg/kg, 0.11 mg/kg, 0.12 mg/kg, 0.13 mg/kg, 0.14 mg/kg, 0.15 mg/kg, 0.16 mg/kg, 0.17 mg/kg, 0.18 mg/kg, 0.19 mg/kg, 0.2 mg/kg at an interval of 1 week or 10 days.

In another exemplary embodiment, the fusion protein is administered at a dose of 0.1 mg/kg, 0.11 mg/kg, 0.12 mg/kg, 0.13 mg/kg, 0.14 mg/kg, 0.15 mg/kg, 0.16 mg/kg, 0.17 mg/kg, 0.18 mg/kg, 0.19 mg/kg, 0.2 mg/kg, 0.21 mg/kg, 0.22 mg/kg, 0.23 mg/kg, 0.24 mg/kg, 0.25 mg/kg, 0.26 mg/kg, 0.27 mg/kg, 0.28 mg/kg, 0.29 mg/kg, or 3 mg/kg at an interval of 2 weeks, or with a frequency of once every other week, twice per month, or three times per month.

In an embodiment, the administration may be carried out parentally, for example subcutaneously.

Various aspects will now be described only by way of non-limiting example with reference to the following Examples.

### Preparation Example 1 Preparation of GLP-1-hyFc5, GLP-1-hyFc9, GLP-1-hyFc8, and GLP-1-hyFc11

By following the process described in Example 1-1 of U.S. Pat. No. 10,538,569, of which content is incorporated herein by reference, GLP-1-hyFc5 fusion polypeptide (SEQ ID NO: 54), GLP-1-hyFc9 (SEQ ID NO: 41), GLP-1-hyFc8 (SEQ ID NO: 40), and GLP-1-hyFc11 (SEQ ID NO: 42) fusion polypeptides.

Single clone selection of transfected cells and purification of secreted proteins were conducted in a similar way to the protocol in case of other hybrid Fc fused recombinant protein as previously described^{17,18}. Dulaglutide (TRULICITY®) was purchased from Elli Lilly and LiCl for CTA study was purchased from Sigma Aldrich (USA).

### Example 1

Materials

GLP-1(A2G)-hyFc9 (SEQ ID NO: 41) obtained in Preparation Example 1 was used as the GPL-1-gFc of Formula (I). In

Analysis of Potency in Cell Based In-Vitro Activity

In order to evaluate the potency of test articles (TA), the degree of cyclic AMP induction by GLP-1 specific response, a transgenic cell line (GLP1R_cAMP/luc) was constructed to express GLP-1 receptor in cAMP-specific luciferin-expressing cell lines. After thawing and appropriate maintaining, 2×10⁵ cells/mL cells with growth media (90% DMEM/High glucose, 10% FBS, 130 ug/mL Hygromycin B God, 5 ug/mL Puromycin) were seeded in T-75 flask and incubated in CO₂ incubator at 37° C. until 70-80% confluence. When the confluency of cells reach the 70-80%, cells were washed with PBS and 0.05% TE (Trypsin EDTA) was added to separate the cells from the flask. The cells were collected and washed as needed for activity evaluation and diluted with 0.5% FBS and DMEM/High glucose media to seed cells at 2×10⁴ cells/80 uL/well. After incubating the cells for about 16 hours in CO₂ incubator at 37° C., 20 uL/well of TAs with various concentrations were treated and reacted in CO₂ incubator at 37° C. for 5 hours. The Bright-Glo™ assay reagent (Promega, USA) was treated with 100 uL/well and reacted at room temperature for 2 minutes. After the reaction, the luminescence was measured by using a luminometer (BioTek, USA).

Analysis of Binding Affinity by SPR (Surface Plasmon Resonance)

Binding affinity of each TAs were evaluated by SPR (Proteon XPR36, BIO-RAD) and based on the protocol modified from general procedure of SPR analysis in published paper. Specifically, the proteon GLC chip (BIO-RAD, USA) was stabilized with PBST (PBS+0.01% tween 20, pH 7.4). Stabilized GLC chip was activated with 150 uL of Sulfo-NHS (0.001 M) and EDC (0.04M) (1:1) followed by immobilization of 10 ug/mL human GLP-1 receptor (Abcam, UK) diluted in acetate buffer (pH 5.0). After immobilization level was recorded and deactivation by 1 M Ethanolamine-HCl (pH 8.5), different concentrations of Dulaglutide and GLP-1-gFc (0, 1.25, 2.5, 5, 10 uM) were injected to each channel of chip. Chip was regenerated by 25 mM NaOH and checked ‘Zero base’ before repeated analysis of same molecule or other TA. All binding sesorgrams were collected, processed, and analyzed using the integrated Proteon Manager software (Bio-RAD, USA). Binding curves were fitted using the Laugmuir model.

Animals

All animal studies were conducted according to the protocols approved by the Institutional Animal Care and Use Committee at Genexine (Korea), or Wuxi apptec (China). Obese (C57BL/6J-ob/ob) mice and DBA/2 mice were obtained from SLC (Japan) and Koatech (Korea), respectively. Obtained mice were housed in an appropriate number per cage on a 12 h/12 h light-dark cycle at 20±2° C. Sterilized solid animal feed with radiation (Teklad certified irradiated global 18% protein diet, 2918C, Harlan Co., Ltd., US) and sterilized water were fed freely using appropriate dispenser and bottle.

Male Cynomolgus monkeys were obtained from Hainan Jingang Biotech (China) and individually housed in stainless steel cages of animal facility of Wuxi apptech. Animals were supplied with monkey feed twice daily and reverse-osmosis purified and chlorinated water ad libitum by an automated system. Monkeys for ECG study were instrumented with transmitters (DSI TL11M2-D70-PCT) according to Wuxi's SOPs and only individual exhibiting normal ECG parameters were enrolled into the study.

Dose-Finding of GLP-1-gFc in Db/Db Mice

Male diabetic (5 weeks old, db/db) mice were acclimated to feeding environment for 1 weeks. Blood glucose in non-fasting status was measured to allocate animals to treatment groups (n=8/group): vehicle, Dulaglutide 0.6 mg/kg, GLP-1-gFc 0.6 mg/kg, and GLP-1-gFc 2.4 mg/kg. All TAs were diluted with dedicated formulation buffers to prepare drug product for injection and analyzed with GLP-1 ELISA that detect active form of GLP-1 where mouse anti-human IgG4 (BD bioscience, USA) and biotinylated n-terminal specific GLP-1 antibody (Thermofisher, USA) were used for capture and detection antibodies, respectively. Analyzed TAs were weekly administered via Subcutaneous (SC) route for 6 weeks. Non-fasting blood glucose was measured once a week during treatment period and glycated hemoglobin (HbA1c) was measured biweekly starting from week 0.

Assessment of Anti-Diabetic/Obesity Effects in Ob/Ob Mice

Female obese (6 weeks old, ob/ob) mice were acclimated to feeding environment and operating procedures such as injection and grasp for 3 weeks. Body weight was measured to allocate animals to treatment groups (n=8/group): vehicle, Dulaglutide 0.6 mg/kg, GLP-1-gFc 2.4 mg/kg. All TAs were diluted and analyzed with active GLP-1 ELISA as described above and administered weekly via Subcutaneous (SC) route for 4 weeks. Food intake and Body weight were measured once a week during treatment period and glycated hemoglobin (HbA1c) was measured at start (week 0) and the end of the treatment period (week 4).

Conditioned Taste Avoidance (CTA) Study

CTA study to determine the nausea effect of TAs was modified from the protocol previously described¹⁹. Briefly, acclimated male DBA/2J (5 weeks old) mice were housed individually and given 10 min access to a pre-weighed blueberry bar which was then reweighed to measure consumed amount. After 10 min access, animals were allocated to one of the following treatment groups (n=10/groups): vehicle (s.c.), 0.3M LiCl (i.p.), Dulaglutide 0.6 mg/kg (s.c.), and GLP-1-gFc 2.4 mg/kg. Each TAs were administered immediately after the first exposure of blueberry bar to pair new taste of blueberry with nauseous stimulus by TA. The second blueberry bar was exposed to mice after 14-day wash-out period for the exclusion of food intake suppression by GLP-1 derived test article that could affect the consumption of secondly exposed blueberry bar. Thus wash out of TA was evaluated by the normalization of overnight food intake. Degree of CTA response was determined by reduction of bar consumption compared with vehicle group.

Evaluation of QT Interval Changes in Cynomulgus Monkey

Telemetry implanted cynomulgus monkeys were administered single dose of vehicle by SC before assigning to groups with single injections of following TAs: Dulaglutide 0.07 mg/kg (n=3), GLP-1-gFc 0.28 mg/kg (n=2), GLP-1-gFc 1.14 mg/kg (n=2). The dosage of Dulaglutide (0.07 mg/kg) was determined based on the clinical dose of Dulaglutide and typical dose conversion approach using body surface area (1.5 mg/65 kg×3.08)²⁰. The low dose of GLP-1-gFc (0.28 mg/kg) was multiplied by 4 to be an equivalent dose with Dulaglutide and additionally multiplied by 4 for high dose of GLP-1-gFc. Blood pressure and ECG waveforms were recorded from 2 hours before each dose to 24 hours following each dose. ECG for a minimum of 30 seconds was obtained from all monkeys prior to each dose (at least 30 minutes apart) and 2, 4, 8, 12, 16, and 24 hours post dose. Collected ECG data were used to calculate QTc (corrected QT). The QTc was calculated for each 1-minute mean on dosing days using the equation: QTc=QT−β×(RR-500).

### Example 2: Clinical Study in Healthy Subjects

A First-in-Human, phase 1, single ascending dose, randomized, double-blind, placebo-controlled study to assess the safety, tolerability and pharmacokinetics of subcutaneously administered GX-G6 in healthy men was conducted in accordance with the Declaration of Helsinki and Good Clinical Practice. Subjects were provided written informed consent prior to enrolment. 48 healthy male subjects (n=8/cohort, total 6 cohorts, n=6 for active drug and n=2 for placebo) between the ages of 18 and 40 years old were allowed to participate if they had a body mass index (BMI) between 18 and 29.9 kg/m². Exclusion criteria included any clinically significant pancreatic, hepatic, renal, gastrointestinal, cardiovascular, respiratory, hematological, central nervous system diseases or other significant diseases which might influence either the safety of the subject or the absorption, metabolism, excretion of the active agent under investigation. Also subjects with malignancy and substance abuse or addition such as alcohol and drug in the past 3 years were also excluded. 0.01 mg/kg, 0.02 mg/kg, 0.04 mg/kg, 0.08 mg/kg, 0.16 mg/kg, and 0.24 mg/kg of GLP-1-gFc were sequentially administered based on the decision from Safety monitoring committee (SMC) meeting.

The starting dose of 0.01 mg/kg was determined based on the fact that no-observed-adverse-effect-level (NOAEL) from sub-chronic toxicity study in cynomolgus monkeys is 30 mg/kg resulting in human equivalent dose (HED) of 9.75 mg/kg. As a very conservative approach, safety factor of 1000 was applied resulting in maximum required starting dose (MRSD) of 0.00975 mg/kg which is approximately 0.01 mg/kg. The sub-maximum and maximum doses were adopted to check safety profile of GLP-1-gFc when administered at the equivalent efficacy dose, which is 4-fold higher than that of Dulaglutide, with doses in clinical trials of Dulaglutide²¹.

### (2-1) Safety (Especially Pulse Rate, Adverse Events)

Safety was assessed via monitoring adverse events, vital signs (blood pressure, pulse rate, body temperature), 12-lead ECG, physical examination, and laboratory investigations including anti-drug antibody during screening, several times throughout the study and at the follow-up visit.

### (2-2) Pharmacokinetics (PK)

Blood samples for PK analysis were collected by venous puncture or indwelling venous catheter into serum separation tubes at pre-dose and designated time points ranging from 0.25 h to 648 h post-dose. Blood samples were analyzed for GLP-1-gFc concentration in serum using validated ELISA method that detect n-terminally intact GLP-1 and c-terminal end of gFc. The PK parameters were analyzed using non-compartmental methods using Pharsight WinNonlin® Version 12.5. AUCₗₐₛₜ and Cₘₐₓ of GLP-1-gFc were plotted with each doses to assess dose-proportionality.

### (2-3) Oral Glucose Tolerance Test (OGTT)

After overnight fasting, the subjects drank 300 mL of a commercially available OGTT drink containing 75 grams of glucose within 5 min. Blood sample for determination of blood glucose and insulin was taken before and 0.25, 0.5, 1, 1.5 and 2 hours after intake of the glucose solution. Collected samples were analyzed by Photometric assay and Electrochemiluminescence Immunoassay (ECLIA) using Cobas c501 and Cobas e/601 module (Roche Diagnostics, Switzerland), respectively, to obtain the glucose and insulin versus time kinetics. During the test, the subject remained seated.

### (2-4) Statistical Analyses

SPSS 21 (IBM SPSS, Chicago, Ill., USA) was used to exclude outliers and analyze the statistical significance. Data for PK and human study was expressed as mean±SD and others were expressed as mean±SEM. Statistical significance was determined by Student's t test or Mann-Whitney U test for non-parametric approach. Differences were considered statistically significant at P<0.05.

### (2-5) Discussion

(A) GLP-1-gFc Showed Lower In-Vitro Potency than Dulaglutide in a GLP-1R Over-Expressing Cell Line Because of Rapid Dissociation from GLP-1R

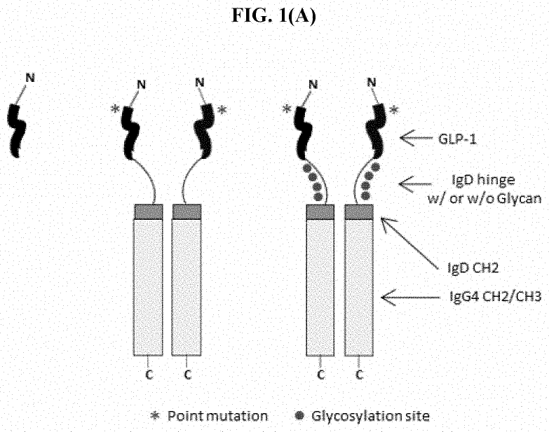

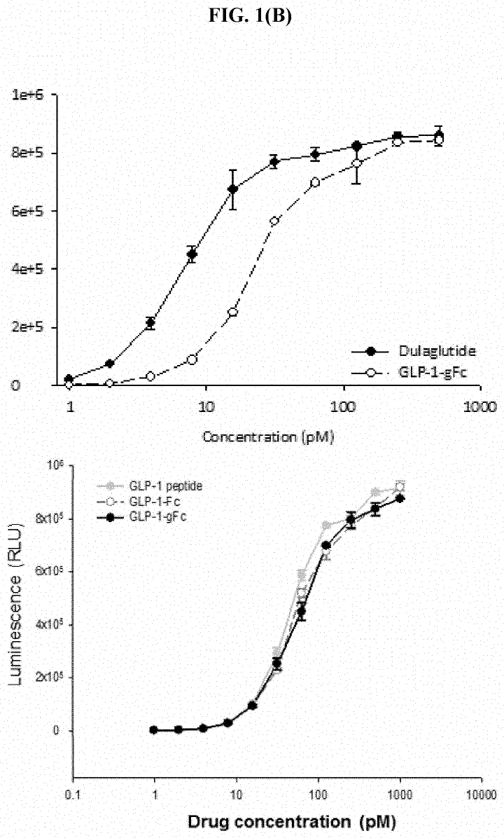

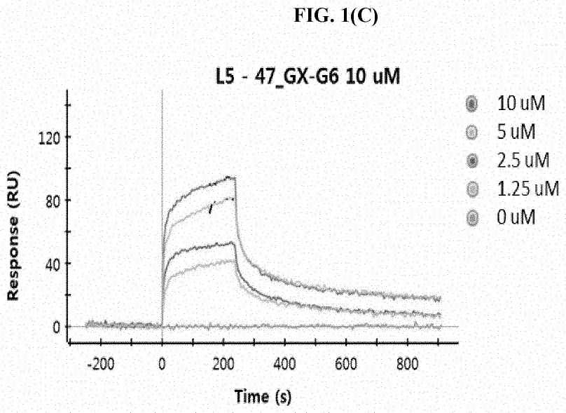

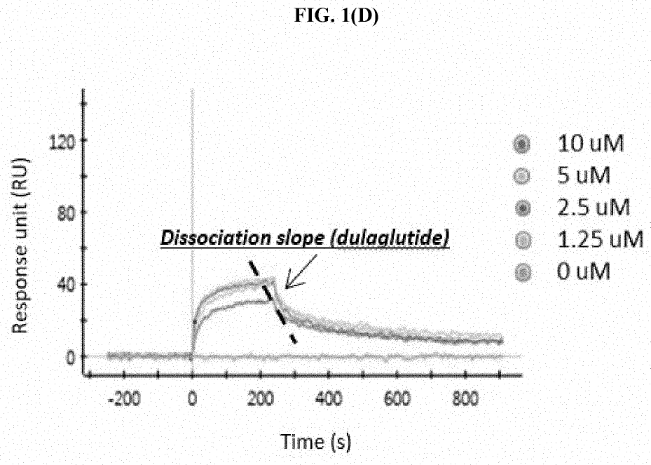

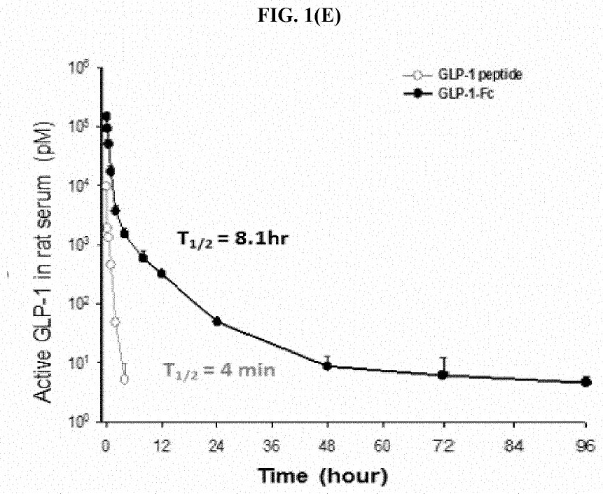

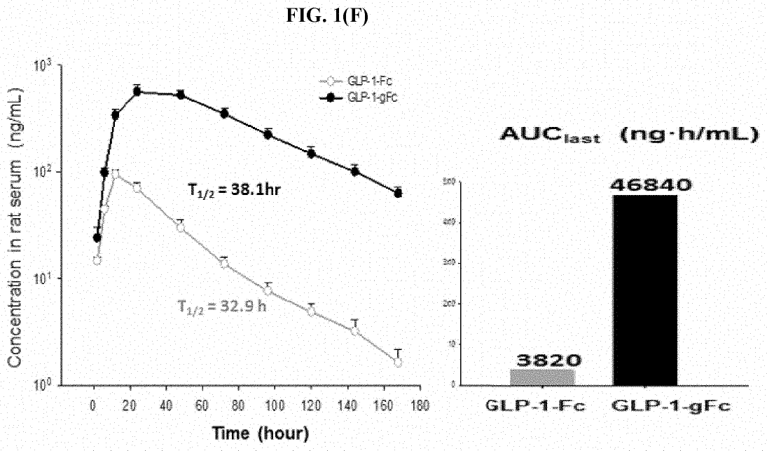

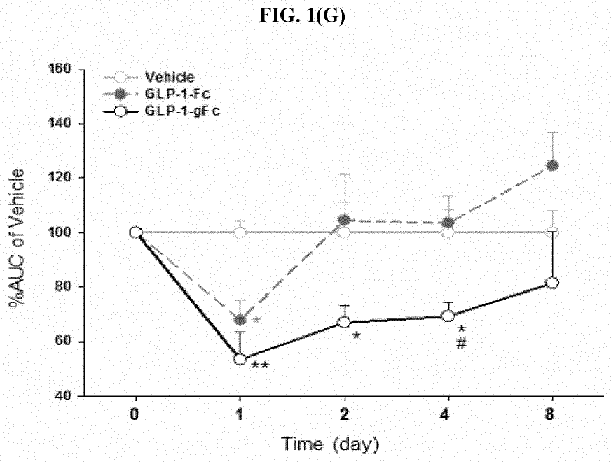

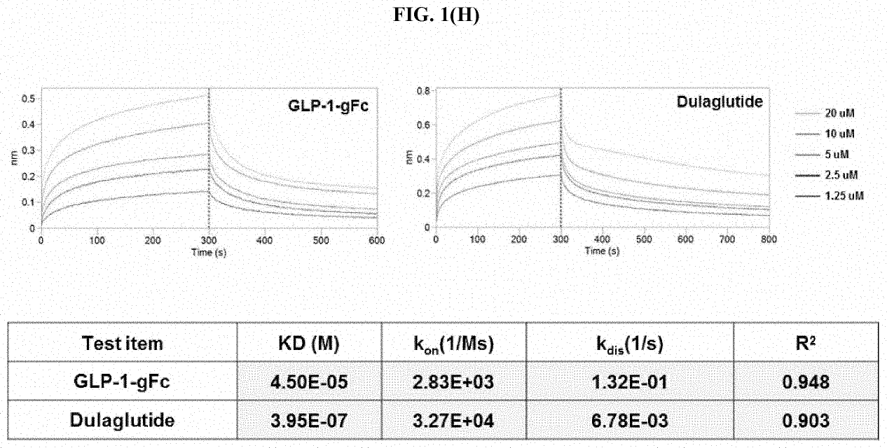

The GLP-1 of the fusion protein of SEQ ID NO: 41 has one point amino acid substitution at n-terminal to prevent enzymatic cleavage by DPP-4²³. Also adoption of 0-glycosylation to IgD hinge region is expected to improve the in-vivo stability without loss of activity. Actually, introduction of O-glycosylation to hinge region showed dramatic enhancement of pharmacokinetics and pharmacodynamics in rodent without loss of activity. See FIGS. 1(A)-1(H). When the two molecules, GLP-1-gFc of Preparation Example 1 and Dulaglutide, were analyzed in the cell based assay using GLP-1 receptor over-expressing cell lines releasing cAMP-dependent luciferin. When the same molar concentration of two molecules were incubated with cell lines, distinctive response curve from each molecules were obtained.

GLP-1-gFc showed relatively lower response at the same molar concentration than Dulaglutide showing 3.5 fold lower EC₅₀ of 23.33 pM (vs 6.66 pM for Dulaglutide) (FIG. 1(B)). To identify the reason for different in-vitro activity of these molecules, binding affinity was evaluated using SPR by flowing them through the human GLP-1 receptor immobilized chip (FIG. 1(C)). GLP-1-gFc and Dulaglutide showed dose-dependent increase of response unit (RU) and GLP-1-gFc showed more rapid decrease of RU than Dulaglutide. The slope of dissociation, represented as dissociation constant (Kd), in GLP-1-gFc was 6.43×10⁻² which is around 10-fold higher than that of Dulaglutide. But association constant (Ka) of GLP-1-gFc was 4.02×10³ which is only 1.7-fold differences with Dulaglutide. This lower binding affinity of GLP-1-gFc was confirmed in BLI (Biolayer Interferometry) system which is different format of analysis to identify binding affinity of molecules. See, FIG. 1(H). Collectively, equilibrium dissociation constant (KD) of GLP-1-gFc and Dulaglutide were 1.6×10⁻⁵ and 9.04×10⁻⁷, respectively, indicating more rapid dissociation of GLP-1-gFc than Dulaglutide from GLP-1 receptor. These observations suggest that GLP-1-gFc has lower binding affinity and in-vitro potency than Dulaglutide because of different structural characteristics.

(B) GLP-1-gFc Shows Comparable Efficacy on Glucose Lowering at 4-Fold Higher Dose than Dulaglutide in Diabetic Db/Db Mice

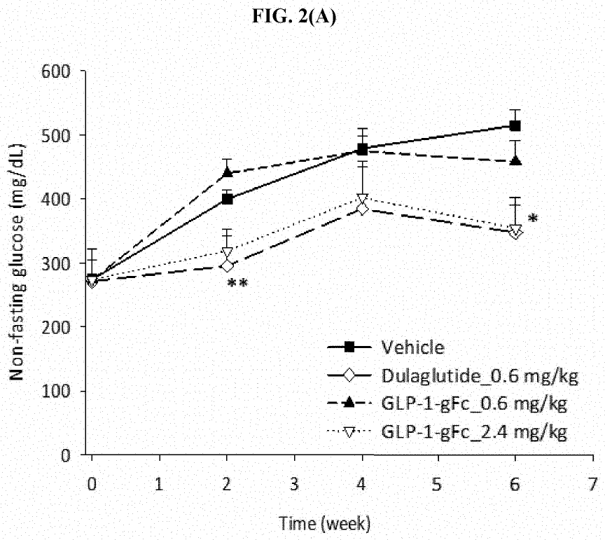

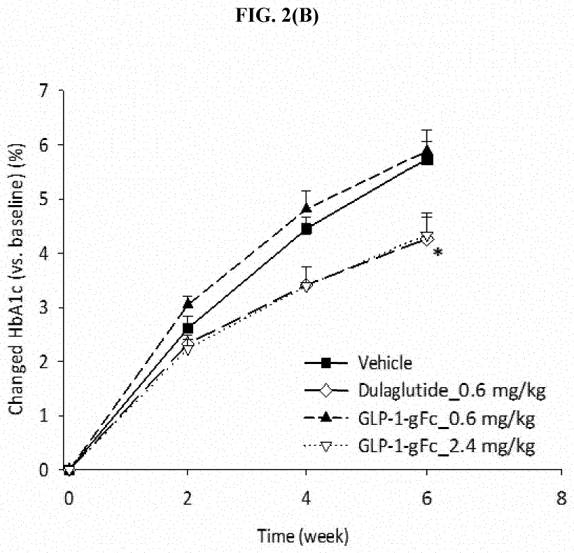

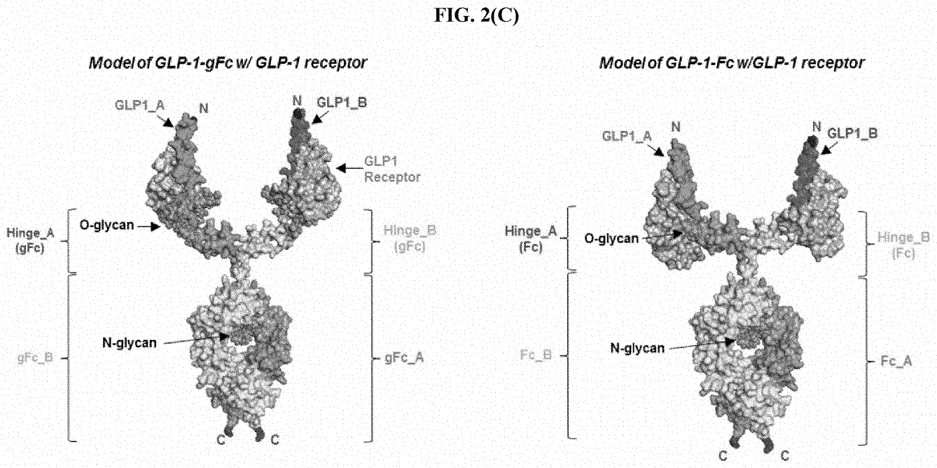

To find the dose that showing comparable anti-diabetic effect, 0.6 mg/kg and 2.4 mg/kg of GLP-1-gFc (GLP-1(A2G)-hyFc9) were evaluated with optimal dose, 0.6 mg/kg, of Dulaglutide in db/db mice^{22,24} (FIGS. 2(A) and 2(B)). GLP-1-gFc and Dulaglutide were administered weekly via SC route for 6 weeks. Non-fasted glucose of vehicle treated group increased from 274 mg/dL to 515 mg/dL (Δglucose: 241 mg/dL) by the end of the study.

All TA treated groups showed statistically significant decrease of terminal glucose level compared with vehicle treated group. Dulaglutide significantly prevented increase of non-fasted glucose level with terminal glucose level of 348 mg/dL (Δglucose: 76.3 mg/dL). And GLP-1-gFc showed dose-dependent effect on delay of glucose increase with terminal glucose level of 459 mg/dL and 355 mg/dL for 0.6 mg/kg and 2.4 mg/kg (Δglucose of 185 mg/dL and 80.1 mg/dL), respectively (FIG. 2(A)). Similar patterns of efficacy were confirmed in glycated hemoglobin (HbA1c) changes. Only Dulaglutide and high dose GLP-1-gFc displayed meaningful reduction of terminal HbA1c (%) after 6-week administration with mean value of 4.26% and 4.34%, respectively (FIG. 2(B)). These results collectively indicated that around 4-fold higher amount of GLP-1-gFc may be required for equivalent anti-diabetic efficacy to Dulaglutide in vivo.

(C) GLP-1-gFc Shows Comparable Efficacy on Glucose Lowering but Weaker Effect on Food Intake and Body Weight Decrease than Dulaglutide

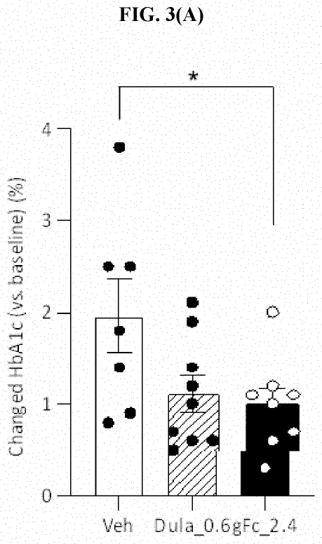

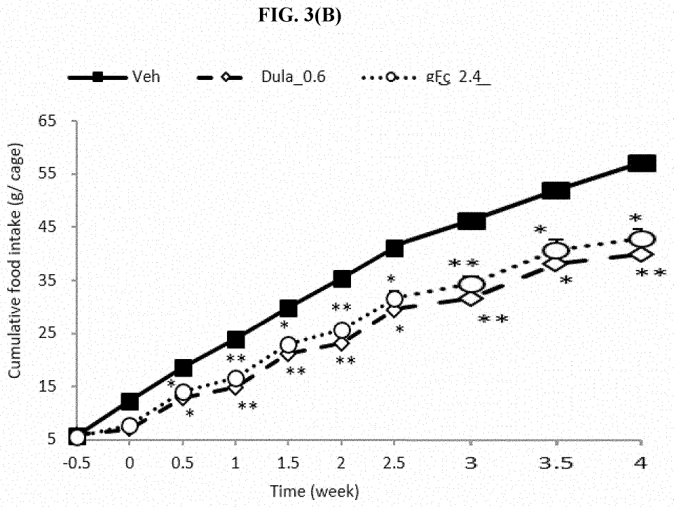

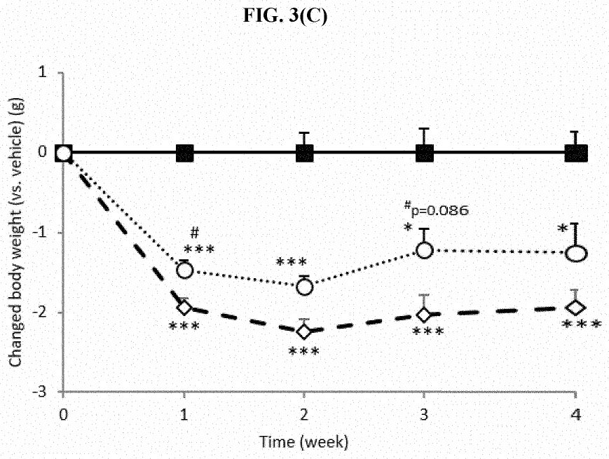

GLP-1 is well known as a pleiotropic ligand having its receptors in various organs such as pancreas, heart, vagus nerve, brain, etc²⁵⁻²⁷. Also reduction of food intake/body weight and secretion of insulin are well known action of GLP-1 caused by signaling of GLP-1 receptor in vagal nerve/brain and pancreas²⁸. To further investigate and compare the effect of GLP-1-gFc and Dulaglutide on GLP-1 receptors in pancreas and vagus nerve/brain, GLP-1-gFc and Dulaglutide were administered weekly via Subcutaneous (SC) route to obese ob/ob mice for 4 weeks. Both GLP-1-gFc and Dulaglutide significantly delayed increase of HbA1c (%) (Δ HbA1c) compared with Vehicle (0.9%, 1.1% and 2.0% for GLP-1-gFc, Dulaglutide and vehicle) (FIG. 3(A)). Dulaglutide decreased cumulative food intake and body weight significantly compared with vehicle (−17 g/cage and −1.9% vs. vehicle). On the other hand, GLP-1-gFc showed weaker response on these two parameters where the difference between 2.5 mg/kg of GLP-1-gFc and Dulaglutide were significant at week 2 and 3 in body weight change (FIG. 3(B) and FIG. 3(C)). These findings suggested that Dulaglutide and GLP-1-gFc could represent different receptor mediated response dependent on the organs that express GLP-1 receptors in different levels.

(D) Equivalent Dose of GLP-1-gFc has Lesser Nausea/Vomiting Response and Risk of QT Elongation than Dulaglutide

Conditioned Taste Aversion (CTA) study^{7,19} in mice and monitoring of Electrocardiogram (ECG) in monkey were conducted to further investigate the response of GLP-1-gFc of Preparation Example 1 compared with Dulaglutide in extra-pancreatic organs. For CTA study, the blueberry bar was exposed to mice (n=10/groups) right before administration of vehicle, 0.3M LiCl, Dulaglutide 0.6 mg/kg, and GLP-1-gFc 2.4 mg/kg. 2^{nd} consumption of blueberry bar was recorded to evaluate nausea/vomit response by each test molecules which are previously paired with 1^{st} blueberry bar exposure.

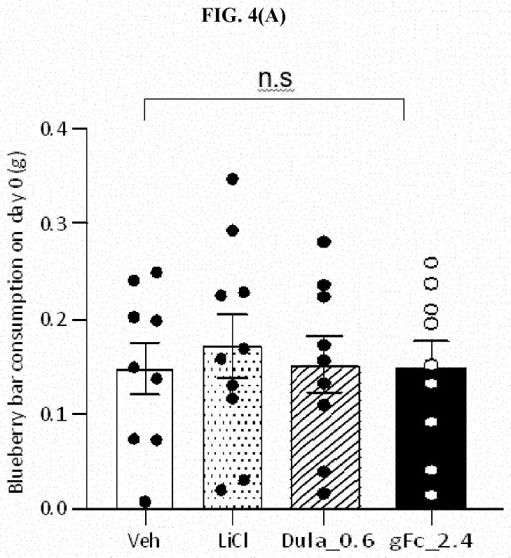

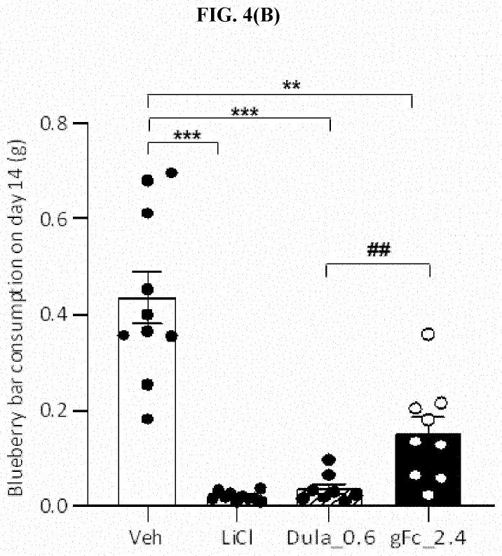

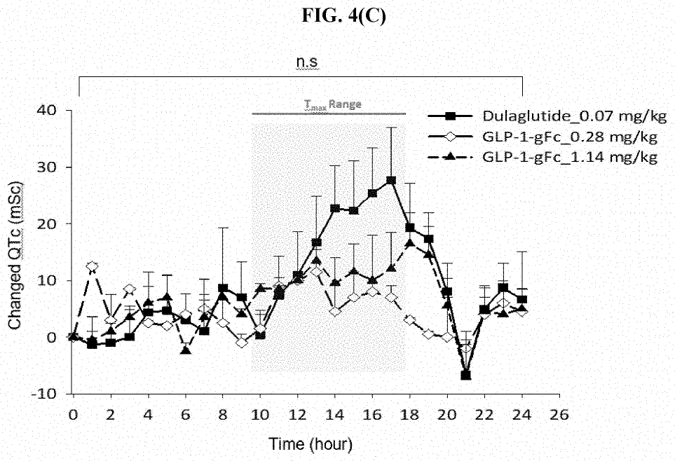

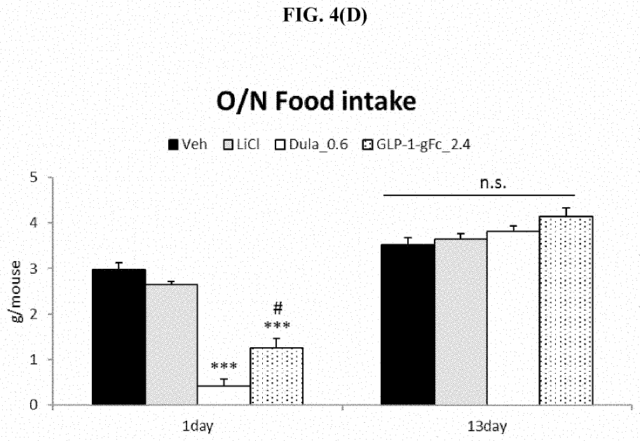

At 1^{st} exposure of blueberry bar, the consumption was almost the same among groups allocated to each test molecules (FIG. 4(A)). But consumption of blueberry bar at 2^{nd} exposure was reduced significantly in LiCl and Dulaglutide paired groups. However, reduction of bar consumption by GLP-1-gFc were much less than LiCl and Dulaglutide groups showing statistical significance with Dulaglutide paired group (FIG. 4(B)). 1 day before 2^{nd} exposure of blueberry bar, overnight food intake was measured to ensure the exclusion of food intake suppression effect by long-acting GLP-1-gFc and Dulaglutide. There was no significant differences between groups in contrast with overnight food intake at day 1 post injection.

As supported by FIG. 4(D), which shows confirmed drug-wash out evaluated by overnight food intake before the second exposure to blueberry bar in CTA study (n=8-10/group), overnight food intake on day 1 post-injection was dramatically reduced in the treated groups which received either dulaglutide or GLP-1-gFc. In contrast, one day before the second exposure (day 13), overnight food intake did not differ between the GLP-1-gFc and dulaglutide groups, confirming complete wash-out of GLP-1-RA-related food intake suppression. Results are presented as means±standard errors of the mean. ***p<0.001 vs. vehicle, # p<0.01 vs. dulaglutide, Mann-Whitney U test. n.s., non-significant; Dula_0.6, dulaglutide 0.6 mg/kg; gFc 2.4, GLP-1-gFc 2.4 mg/kg.

This result demonstrate that the response of GLP-1-gFc to vagal nerve/brain is different from that of Dulaglutide which is inconsistent with the trend observed in pancreas.

To evaluate and compare the cardiovascular effect of GLP-1-gFc and Dulaglutide, total 7 male Telementry implanted Cynomolgus monkeys received 0.07 mg/kg Dulaglutide (n=3), 0.28 mg/kg GLP-1-gFc (n=2), 1.14 mg/kg GLP-1-gFc (n=2) by SC route (FIG. 4(C)). The monkeys received single dose of vehicle followed by 19 days wash-out period and administration of GLP-1-gFc or Dulaglutide. ECG wave forms, heart rate, and blood pressure were recorded from at 2 hours prior to dose to 24 hours post-dose.

Even though there were no treatment-related clinical signs after single administration, numerically meaningful differences between the GLP-1-gFc according to the instant disclosure and Dulaglutide in corrected QT (QTc) interval were identified during the ECG monitoring period. Dulaglutide increased QTc interval at specific time range of 10˜20 hours which is predicted as Tₘₐₓ, whereas low and high dose of GLP-1-gFc did not increased QTc. But these differences did not generated any differences in heart rate and blood pressure.

Collectively these findings suggest that GLP-1-gFc according to the instant disclosure could give relatively milder response to GLP-1 receptors on vagal nerve and heart than receptors on pancreas maybe because of its attenuated receptor affinity. And this phenomena is distinct from other long-acting GLP-1 analogues with high potency like Dulaglutide.

(E) Dose-Dependent, Long-Acting Pharmacokinetics (PK) of GLP-1-gFc Following Single SC Administration to Healthy Subject.

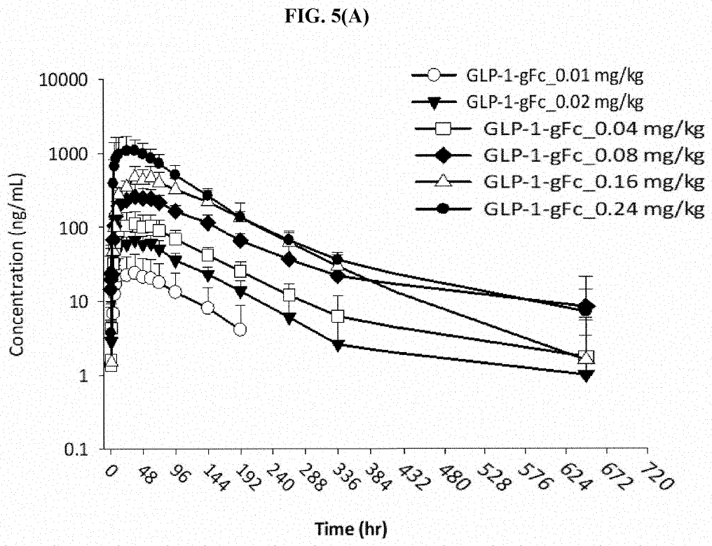

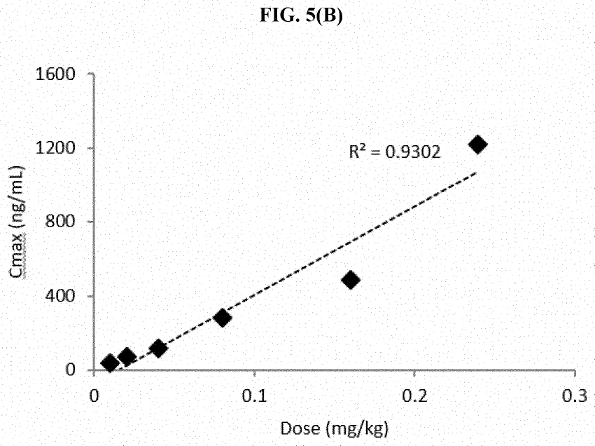

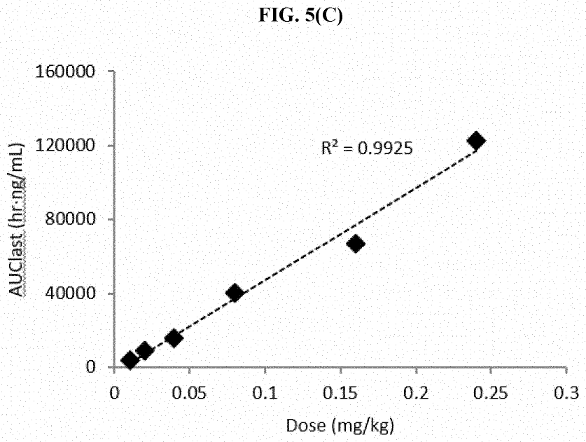

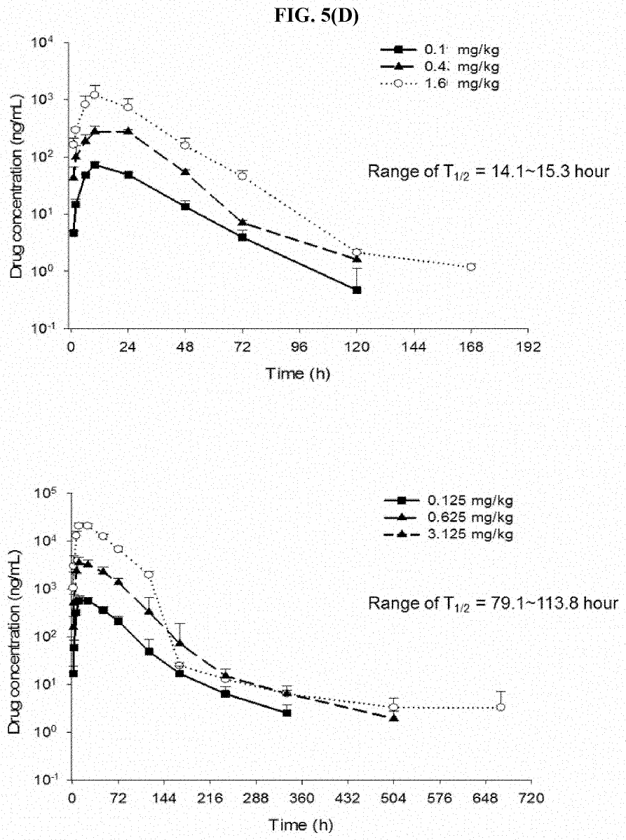

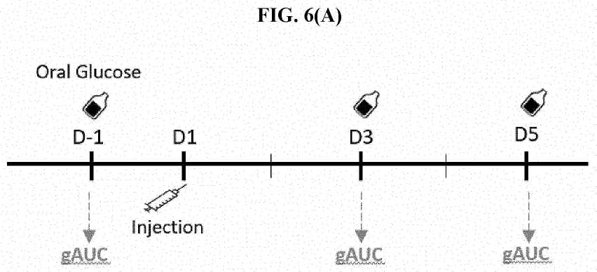

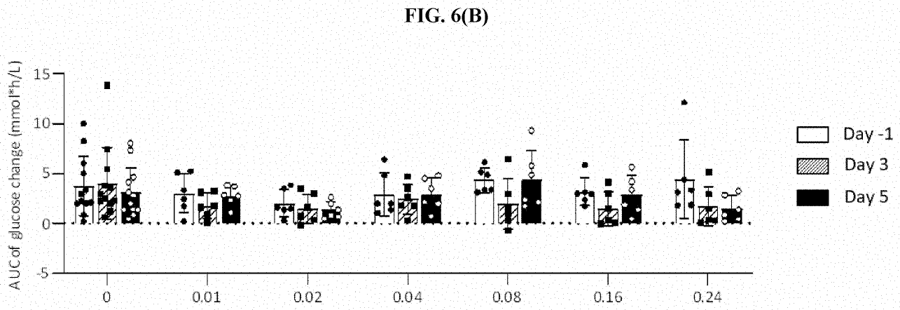

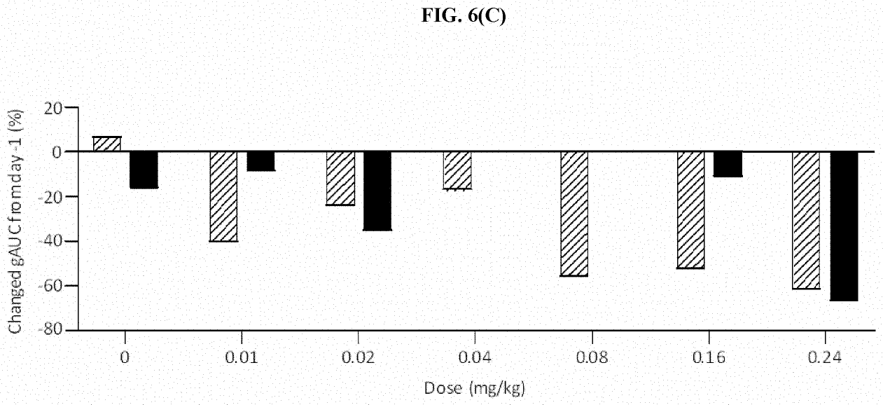

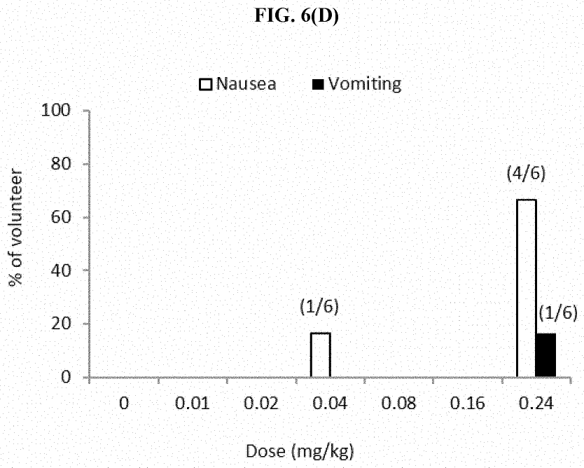

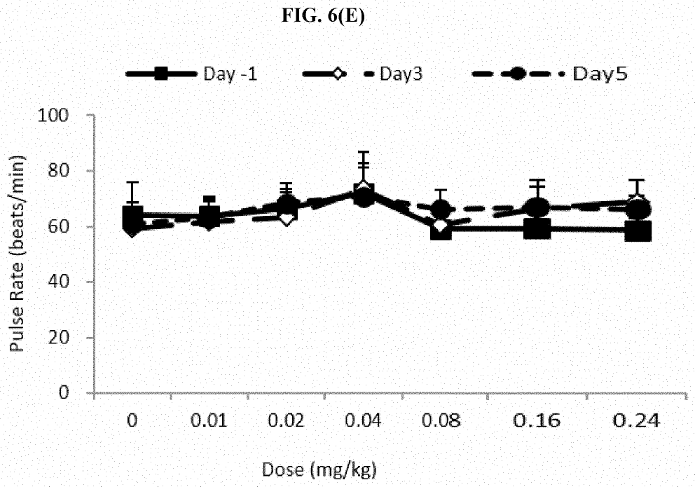

Purified GLP-1-gFc of Preparation Example 1 showed long-acting PK profiles in SD rat and Cynomolgus monkeys with half-life of 14.1-15.3 hours and 79.1-113.8 hours, respectively. Also it enhanced insulin secretion and glucose reduction dose-dependently in diabetic db/db mice, as shown in FIG. 5(D). Based on these results, GLP-1-gFc was administered to healthy man to confirm dose-dependent, long-lasting pharmacokinetics. Increasing 6 different doses from 0.01 mg/kg to 0.24 mg/kg were sequentially administered to healthy subjects and bloods collected at designation time points were analyzed using ELISA method.

The pharmacokinetics of GLP-1-gFc followed a mono-exponential decline with a median T_{1/2} range of 62.5 hours-108 hours through all cohorts (FIG. 5a and Table 2). And geometric mean serum concentration reached their respective peaks at about 36 to 48 hours post-dose with a mean Cₘₐₓ of 36.4 ng/mL (0.01 mg/kg), 68.2 ng/mL (0.02 mg/kg), 102.6 ng/mL (0.04 mg/kg), 242.4 ng/mL (0.08 mg/kg), 454.4 ng/mL (0.16 mg/kg) and 1087.7 ng/mL (0.24 mg/kg). A dose-proportional increase in AUCₗₐₛₜ and Cₘₐₓ was observed for GLP-1-gFc in the plots of Dose versus Cₘₐₓ and AUCₗₐₛₜ which show linearity with R² of 0.9891 and 0.9925, respectively.

(F) GLP-1-gFc was Well Tolerated and had No Remarkable Side Effects on Nausea/Vomiting and Heart Rate Even Though its Good Efficacy in OGTT

Safety and Efficacy of GLP-1-gFc were evaluated based on several safety parameters including blood pressure, pulse rate, Treatment-Emergent Adverse Effect (TEAE) and OGTT per protocol approved by BfArM (The Federal Institute for Drugs and Medical Devices in German). Overall, Single SC doses of GLP-1-gFc in the dose range of 0.01 to 0.24 mg/kg were safe and well tolerated with no generation of antibodies against GLP-1-gFc. There were no SAEs (Severe Adverse Effects) and all TEAEs were of mild to moderate intensity which were resolved by the end of the study.

In the OGTT study, GLP-1-gFc decreased gAUC (AUC in Δglucose vs. time plot) in dose-dependent manner. This decrease was more definite at 3 days post-dose than that at 5 days post-dose in consistent with Tₘₐₓ of GLP-1-gFc of 36-48 hours in pharmacokinetics. Suppression of gAUC in the highest dose (0.24 mg/kg) at day 3 post-dose (approximately −65% from baseline) was the most significant and changes of gAUC in 0.08 mg/kg and 0.16 mg/kg were also remarkable with 55% and 53% suppression from baseline, respectively. Off target effect of GLP-1-gFc was assessed by % of subjects who experienced nausea/vomiting during the study period and purse rate at the same day with in OGTT evaluation. Almost no subjects suffered nausea/vomiting until doses at 0.16 mg/kg with only one subject had nausea at 0.04 mg/kg. At the highest dose, 4 out of 6 and 1 out of 6 subjects experienced transient nausea and vomiting, respectively. And there was no obvious changes in pulse rate from baseline at day 3 and day 5 post-dose through all cohort.

In conclusion, these results support that GLP-1-gFc of the instant disclosure shows stronger effect in glucose lowering in vivo with significantly reduced side effects of nausea/vomiting and QTc at the equivalent efficacy dose to Dulaglutide.

### REFERENCES

1. Abdul-Ghani, M. & DeFronzo, R. A. Is It Time to Change the Type 2 Diabetes Treatment Paradigm? Yes! GLP-1 RAs Should Replace Metformin in the Type 2 Diabetes Algorithm. 40, 1121-1127 (2017).
2. Brown, E., Cuthbertson, D. J. & Wilding, J. P. Newer GLP-1 receptor agonists and obesity-diabetes. Peptides 100, 61-67 (2018).
3. Dhir, G. & Cusi, K. Glucagon like peptide-1 receptor agonists for the management of obesity and non-alcoholic fatty liver disease: a novel therapeutic option. Journal of investigative medicine: the official publication of the American Federation for Clinical Research 66, 7-10 (2018).
4. Ban, K., et al. Cardioprotective and vasodilatory actions of glucagon-like peptide 1 receptor are mediated through both glucagon-like peptide 1 receptor-dependent and -independent pathways. Circulation 117, 2340-2350 (2008).
5. (7) Approaches to glycemic treatment. Diabetes care 38 Suppl, S41-48 (2015).
6. Lorenz, M., et al. Differential effects of glucagon-like peptide-1 receptor agonists on heart rate. Cardiovasc Diabetol 16, 6-6 (2017).
7. Kanoski, S. E., Rupprecht, L. E., Fortin, S. M., De Jonghe, B. C. & Hayes, M. R. The role of nausea in food intake and body weight suppression by peripheral GLP-1 receptor agonists, exendin-4 and liraglutide. Neuropharmacology 62, 1916-1927 (2012).
8. Sikirica, M. V., et al. Reasons for discontinuation of GLP1 receptor agonists: data from a real-world cross-sectional survey of physicians and their patients with type 2 diabetes. Diabetes, metabolic syndrome and obesity: targets and therapy 10, 403-412 (2017).
9. Divino, V., et al. Glucagon-like Peptide-1 receptor agonist treatment patterns among type 2 diabetes patients in six European countries. Diabetes therapy: research, treatment and education of diabetes and related disorders 5, 499-520 (2014).
10. Dalsgaard, N. B. & Vilsboll, T. Effects of glucagon-like peptide-1 receptor agonists on cardiovascular risk factors: A narrative review of head-to-head comparisons. 20, 508-519 (2018).
11. Kang, Y. M. & Jung, C. H. Cardiovascular Effects of Glucagon-Like Peptide-1 Receptor Agonists. Endocrinol Metab 31, 258-274 (2016).
12. Huang, J. H., et al. Glucagon-like peptide-1 regulates calcium homeostasis and electrophysiological activities of HL-1 cardiomyocytes. Peptides 78, 91-98 (2016).
13. Wallner, M., et al. Exenatide exerts a PKA-dependent positive inotropic effect in human atrial myocardium. Journal of Molecular and Cellular Cardiology 89, 365-375 (2015).
14. Garrido, G., et al. Bivalent binding by intermediate affinity of nimotuzumab: a contribution to explain antibody clinical profile. Cancer biology & therapy 11, 373-382 (2011).
15. Ramakrishnan, M. S., et al. Nimotuzumab, a promising therapeutic monoclonal for treatment of tumors of epithelial origin. mAbs 1, 41-48 (2009).
16. Takeda, M., Okamoto, I., Nishimura, Y. & Nakagawa, K. Nimotuzumab, a novel monoclonal antibody to the epidermal growth factor receptor, in the treatment of non-small cell lung cancer. Lung Cancer (Auckland, N. Z.) 2, 59-67 (2011).
17. Lee, J.-H., et al. The pharmacology study of a new recombinant TNF receptor-hyFc fusion protein. Biologicals 41, 77-83 (2013).
18. Im, S. J., et al. Natural Form of Noncytolytic Flexible Human Fc as a Long-Acting Carrier of Agonistic Ligand, Erythropoietin. PLOS ONE 6, e24574 (2011).
19. Swank, M. W. & Sweatt, J. D. Increased histone acetyltransferase and lysine acetyltransferase activity and biphasic activation of the ERK/RSK cascade in insular cortex during novel taste learning. The Journal of neuroscience: the official journal of the Society for Neuroscience 21, 3383-3391 (2001).
20. Nair, A. B. & Jacob, S. A simple practice guide for dose conversion between animals and human. J Basic Clin Pharm 7, 27-31 (2016).
21. Barrington, P., et al. LY2189265, a long-acting glucagon-like peptide-1 analogue, showed a dose-dependent effect on insulin secretion in healthy subjects. Diabetes, obesity & metabolism 13, 434-438 (2011).
22. Glaesner, W., et al. Engineering and characterization of the long-acting glucagon-like peptide-1 analogue LY2189265, an Fc fusion protein. Diabetes/Metabolism Research and Reviews 26, 287-296 (2010).
23. Holst, J. J. The physiology of glucagon-like peptide 1. Physiological reviews 87, 1409-1439 (2007).
24. Kimura, T., et al. Durability of protective effect of dulaglutide on pancreatic β-cells in diabetic mice: GLP-1 receptor expression is not reduced despite long-term dulaglutide exposure. Diabetes & Metabolism 44(2018).
25. Muskiet, M. H. A., et al. GLP-1 and the kidney: from physiology to pharmacology and outcomes in diabetes. Nature reviews. Nephrology 13, 605-628 (2017).
26. Korner, M., Stockli, M., Waser, B. & Reubi, J. C. GLP-1 receptor expression in human tumors and human normal tissues: potential for in vivo targeting. Journal of nuclear medicine: official publication, Society of Nuclear Medicine 48, 736-743 (2007).
27. Pyke, C., et al. GLP-1 receptor localization in monkey and human tissue: novel distribution revealed with extensively validated monoclonal antibody. Endocrinology 155, 1280-1290 (2014).
28. Meier, J. J. GLP-1 receptor agonists for individualized treatment of type 2 diabetes mellitus. Nature reviews. Endocrinology 8, 728-742 (2012).
29. Strohl, W. R. Fusion Proteins for Half-Life Extension of Biologics as a Strategy to Make Biobetters. BioDrugs 29, 215-239 (2015).
30. Hummer, B. T. PHARMACOLOGY/TOXICOLOGY BLA REVIEW AND EVALUATION (Dulaglutide). (2013).
31. Wange, R. L. PHARMACOLOGY/TOXICOLOGY NDA/BLA REVIEW AND EVALUATION (Albiglutide). (2013).
32. Alters, S. E., et al. GLP2-2G-XTEN: a pharmaceutical protein with improved serum half-life and efficacy in a rat Crohn's disease model. PLoS One 7, e50630 (2012).
33. Yang, S. H., Yang, S. I. & Chung, Y. K. A long-acting erythropoietin fused with noncytolytic human Fc for the treatment of anemia. Archives of pharmacal research 35, 757-759 (2012).
34. Glaesner, W., et al. Engineering and characterization of the long-acting glucagon-like peptide-1 analogue LY2189265, an Fc fusion protein. Diabetes/metabolism research and reviews 26, 287-296 (2010).
35. Knudsen, L. B., Hastrup, S., Underwood, C. R., Wulff, B. S. & Fleckner, J. Functional importance of GLP-1 receptor species and expression levels in cell lines. Regulatory peptides 175, 21-29 (2012).

What is claimed is:

1. A method for regulating blood glucose level in a subject in need thereof, comprising administering to the subject an effective amount of a fusion peptide comprising (a) glucagon-like peptide-1 (GLP-1) peptide and (b) an immunoglobulin Fc region,
wherein the immunoglobulin Fc region (b) comprises
(i) an isolated IgD hinge region consisting of 35 to 49 consecutive amino acid residues from the C-terminus of SEQ ID NO: 3; and
(ii) a CH2 domain and a CH3 domain of the immunoglobulin Fc polypeptide.

2. The method of claim 1, wherein the effective amount ranges from about 0.01 mg/kg to about 1 mg/kg body weight.

3. The method of claim 1, wherein the fusion peptide is administered parentally at an interval of 1 week or greater.

4. The method of claim 1, wherein the subject suffers from diabetes, glucose intolerance, and/or insulin resistance.

5. The method of claim 1, wherein the GLP-1 peptide (a) comprises the amino acid sequence selected from the group consisting of SEQ ID NO: 1 and SEQ ID NOS: 10 to 34.

6. The method of claim 1, wherein the isolated IgD hinge region (i) comprises the amino acid sequence of SEQ ID NO: 36, 37, or 38.

7. The method of claim 1, wherein the immunoglobulin Fc region (b) comprises the amino acid sequence selected from the group consisting of SEQ ID NOS: 4 to 8.

8. The method of claim 1, wherein the fusion peptide comprises the amino acid sequence selected from the group consisting of SEQ ID NOS: 40 to 42 and 54.

9. The method of claim 3, wherein the fusion peptide is administered at a dose of 0.01 mg/kg to 0.2 mg/kg at an interval of 1 week or at a frequency of once per week.

10. The method of claim 3, wherein the fusion peptide is administered at a dose of 0.2 mg/kg to 0.5 mg/kg at an interval of 2 weeks, or at a frequency of every other week.

11. The method of claim 1, wherein the subject suffers from diabetes.

12. The method of claim 11, wherein the diabetes is type II diabetes.

13. The method of claim 3, wherein the fusion peptide is administered subcutaneously.

14. The method of claim 1, wherein the fusion peptide is a dimer comprising two peptides joined together by sulfide bonds wherein the each peptide comprises the Fc region (b) of SEQ ID NO: 4, 5, 6, 7, or 8.

15. A method for preventing and/or treating diabetes in a subject in need thereof, administering to the subject an effective amount of a fusion peptide comprising (a) glucagon-like peptide-1 (GLP-1) peptide and (b) an immunoglobulin Fc region,
wherein the immunoglobulin Fc region (b) comprises
(i) an isolated IgD hinge region consisting of 35 to 49 consecutive amino acid residues from the C-terminus of SEQ ID NO: 3; and
(ii) a CH2 domain and a CH3 domain of the immunoglobulin Fc polypeptide.

16. The method of claim 15, wherein the effective amount ranges from about 0.01 mg/kg to about 1 mg/kg body weight.

17. The method of claim 15, wherein the fusion peptide is administered parentally at an interval of 1 week or greater.

18. The method of claim 15, wherein the fusion peptide is administered at a dose of 0.01 mg/kg to 0.2 mg/kg at an interval of 1 week or at a frequency of once per week.

19. The method of claim 15, wherein the fusion peptide is administered at a dose of 0.2 mg/kg to 0.5 mg/kg at an interval of 2 weeks, or at a frequency of every other week.

20. The method of claim 15, wherein the diabetes is non-insulin dependent diabetes or insulin dependent diabetes.


SEQUENCE LISTING (54 sequences)

SEQ ID NO: 1 - protein, 31 aa, Homo sapiens
  GLP-1(7-37)
HAEGTFTSDVSSYLEGQAAKEFIAWLVKGRG

SEQ ID NO: 2 - protein, 384 aa, Homo sapiens
  amino acid sequence of human IgD constant region (Genbank accession No. P01880)
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 3 - protein, 327 aa, Homo sapiens
  amino acid sequence of Partial human IgG4 constant region (Genbank accession No. AAH25985)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 245 aa, Artificial Sequence
  amino acid sequence of hyFc
RNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 245 aa, Artificial Sequence
  amino acid sequence of hyFcM1
RNTGRGGEEKKGGKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 245 aa, Artificial Sequence
  amino acid sequence of hyFcM2
RNTGRGGEEKKGSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 245 aa, Artificial Sequence
  amino acid sequence of hyFcM3
RNTGRGGEEKKSGKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 245 aa, Artificial Sequence
  amino acid sequence of hyFcM4
RNTGRGGEEKKSSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 243 aa, Artificial Sequence
  amino acid sequence of mouse IgG Fc variant
ASAEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGNSGS

SEQ ID NO: 10 - protein, 39 aa, Homo sapiens
  Exendin-4
HGEGTFTSDLSKQMEEEAVRLFIEWLKNGGPSSGAPPPS

SEQ ID NO: 11 - protein, 30 aa, Homo sapiens
  GLP-1(7-36)
HAEGTFTSDVSSYLEGQAAKEFIAWLVKGR

SEQ ID NO: 12 - protein, 31 aa, Artificial Sequence
  GLP-1(7-37) analogue (A8G)
HGEGTFTSDVSSYLEGQAAKEFIAWLVKGRG

SEQ ID NO: 13 - protein, 31 aa, Artificial Sequence
  GLP-1(7-37) analogue (A8V)
HVEGTFTSDVSSYLEGQAAKEFIAWLVKGRG

SEQ ID NO: 14 - protein, 31 aa, Artificial Sequence
  GLP-1(7-37) analogue (G22E)
HAEGTFTSDVSSYLEEQAAKEFIAWLVKGRG

SEQ ID NO: 15 - protein, 31 aa, Artificial Sequence
  GLP-1(7-37) analogue (R36G)
HAEGTFTSDVSSYLEGQAAKEFIAWLVKGGG

SEQ ID NO: 16 - protein, 31 aa, Artificial Sequence
  GLP-1(7-37) analogue (A8G/G22E)
HGEGTFTSDVSSYLEEQAAKEFIAWLVKGRG

SEQ ID NO: 17 - protein, 31 aa, Artificial Sequence
  GLP-1(7-37) analogue (A8V/G22E)
HVEGTFTSDVSSYLEEQAAKEFIAWLVKGRG

SEQ ID NO: 18 - protein, 31 aa, Artificial Sequence
  GLP-1(7-37) analogue (A8G/R36G)
HGEGTFTSDVSSYLEGQAAKEFIAWLVKGGG

SEQ ID NO: 19 - protein, 31 aa, Artificial Sequence
  GLP-1(7-37) analogue (A8V/R36G)
HVEGTFTSDVSSYLEGQAAKEFIAWLVKGGG

SEQ ID NO: 20 - protein, 31 aa, Artificial Sequence
  GLP-1(7-37) analogue (G22E/R36G)
HAEGTFTSDVSSYLEEQAAKEFIAWLVKGGG

SEQ ID NO: 21 - protein, 31 aa, Artificial Sequence
  GLP-1(7-37) analogue (A8G/G22E/R36G)
HGEGTFTSDVSSYLEEQAAKEFIAWLVKGGG

SEQ ID NO: 22 - protein, 31 aa, Artificial Sequence
  GLP-1(7-37) analogue (A8V/G22E/R36G)
HVEGTFTSDVSSYLEEQAAKEFIAWLVKGGG

SEQ ID NO: 23 - protein, 30 aa, Homo sapiens; identical to SEQ ID NO: 11

SEQ ID NO: 24 - protein, 30 aa, Artificial Sequence
  GLP-1(7-36) analogue (A8G)
HGEGTFTSDVSSYLEGQAAKEFIAWLVKGR

SEQ ID NO: 25 - protein, 30 aa, Artificial Sequence
  GLP-1(7-36) analogue (A8V)
HVEGTFTSDVSSYLEGQAAKEFIAWLVKGR

SEQ ID NO: 26 - protein, 30 aa, Artificial Sequence
  GLP-1(7-36) analogue (G22E)
HAEGTFTSDVSSYLEEQAAKEFIAWLVKGR

SEQ ID NO: 27 - protein, 30 aa, Artificial Sequence
  GLP-1(7-36) analogue (R36G)
HAEGTFTSDVSSYLEGQAAKEFIAWLVKGG

SEQ ID NO: 28 - protein, 30 aa, Artificial Sequence
  GLP-1(7-36) analogue (A8G/G22E)
HGEGTFTSDVSSYLEEQAAKEFIAWLVKGR

SEQ ID NO: 29 - protein, 30 aa, Artificial Sequence
  GLP-1(7-36) analogue (A8V/G22E)
HVEGTFTSDVSSYLEEQAAKEFIAWLVKGR

SEQ ID NO: 30 - protein, 30 aa, Artificial Sequence
  GLP-1(7-36) analogue (A8G/R36G)
HGEGTFTSDVSSYLEGQAAKEFIAWLVKGG

SEQ ID NO: 31 - protein, 30 aa, Artificial Sequence
  GLP-1(7-36) analogue (A8V/R36G)
HVEGTFTSDVSSYLEGQAAKEFIAWLVKGG

SEQ ID NO: 32 - protein, 30 aa, Artificial Sequence
  GLP-1(7-36) analogue (G22E/R36G)
HAEGTFTSDVSSYLEEQAAKEFIAWLVKGG

SEQ ID NO: 33 - protein, 30 aa, Artificial Sequence
  GLP-1(7-36) analogue (A8G/G22E/R36G)
HGEGTFTSDVSSYLEEQAAKEFIAWLVKGG

SEQ ID NO: 34 - protein, 30 aa, Artificial Sequence
  GLP-1(7-36) analogue (A8V/G22E/R36G)
HVEGTFTSDVSSYLEEQAAKEFIAWLVKGG

SEQ ID NO: 35 - protein, 64 aa, Homo sapiens
RWPESPKAQASSVPTAQPQAEGSLAKATTAPATTRNTGRGGEEKKKEKEKEEQEERETKTPECP

SEQ ID NO: 36 - protein, 35 aa, Homo sapiens
APATTRNTGRGGEEKKKEKEKEEQEERETKTPECP

SEQ ID NO: 37 - protein, 40 aa, Homo sapiens
AKATTAPATTRNTGRGGEEKKKEKEKEEQEERETKTPECP

SEQ ID NO: 38 - protein, 49 aa, Homo sapiens
AQPQAEGSLAKATTAPATTRNTGRGGEEKKKEKEKEEQEERETKTPECP

SEQ ID NO: 39 - protein, 215 aa, Artificial Sequence
  IgD & IgG4 Hybrid CH2 &CH3 region
SHTQPLGVFLFPPKPKDTLMISRTPEVTCVVVDVSQEDPEVQFNWYVDGVEVHNAKTKPREEQFASTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 281 aa, Artificial Sequence
  GLP-1-hyFc8
HGEGTFTSDVSSYLEGQAAKEFIAWLVKGRGAPATTRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 286 aa, Artificial Sequence
  GLP-1-hyFc9
HGEGTFTSDVSSYLEGQAAKEFIAWLVKGRGAKATTAPATTRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 295 aa, Artificial Sequence
  GLP-1-hyFc11
HGEGTFTSDVSSYLEGQAAKEFIAWLVKGRGAQPQAEGSLAKATTAPATTRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 43 - DNA, 93 bp, Homo sapiens
cacgccgaaggcaccttcaccagcgacgtgagcagctacctggaaggccaggctgccaaggagttcatcgcctggctggtgaaaggcagaggc

SEQ ID NO: 44 - DNA, 93 bp, Artificial Sequence
  GLP-1(7-37) analogue (A8G)
cacggcgaaggcaccttcaccagcgacgtgagcagctacctggaaggccaggctgccaaggagttcatcgcctggctggtgaaaggcagaggc

SEQ ID NO: 45 - DNA, 90 bp, Homo sapiens
cacgccgaaggcaccttcaccagcgacgtgagcagctacctggaaggccaggctgccaaggagttcatcgcctggctggtgaaaggcaga

SEQ ID NO: 46 - DNA, 192 bp, Homo sapiens
agatggcccgagagccctaaggcccaggccagctccgtgcccacagctcagccacaggctgagggaagcctcgccaaggcaacgactgcgccggccactacgcgcaacaccggccgcggcggcgaggagaagaagaaggagaaggagaaggaggagcaggaggagcgcgagaccaagacccccgagtgcccc

SEQ ID NO: 47 - DNA, 105 bp, Homo sapiens
gcgccggccactacgcgcaacaccggccgcggcggcgaggagaagaagaaggagaaggagaaggaggagcaggaggagcgcgagaccaagacccccgagtgcccc

SEQ ID NO: 48 - DNA, 120 bp, Homo sapiens
gccaaggcaacgactgcgccggccactacgcgcaacaccggccgcggcggcgaggagaagaagaaggagaaggagaaggaggagcaggaggagcgcgagaccaagacccccgagtgcccc

SEQ ID NO: 49 - DNA, 147 bp, Homo sapiens
gctcagccacaggctgagggaagcctcgccaaggcaacgactgcgccggccactacgcgcaacaccggccgcggcggcgaggagaagaagaaggagaaggagaaggaggagcaggaggagcgcgagaccaagacccccgagtgcccc

SEQ ID NO: 50 - DNA, 645 bp, Artificial Sequence
  IgD & IgG4 Hybrid CH2 & CH3 region
agccacacccagcccctgggcgtgttcctgttcccccccaagcccaaggacaccctgatgatcagccgcacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 51 - DNA, 843 bp, Artificial Sequence
  GLP-1-hyFc8
cacggcgaaggcaccttcaccagcgacgtgagcagctacctggaaggccaggctgccaaggagttcatcgcctggctggtgaaaggcagaggcgcgccggccactacgcgcaacaccggccgcggcggcgaggagaagaagaaggagaaggagaaggaggagcaggaggagcgcgagaccaagacccccgagtgccccagccacacccagcccctgggcgtgttcctgttcccccccaagcccaaggacaccctgatgatcagccgcacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 52 - DNA, 858 bp, Artificial Sequence
  GLP-1-hyFc9
cacggcgaaggcaccttcaccagcgacgtgagcagctacctggaaggccaggctgccaaggagttcatcgcctggctggtgaaaggcagaggcgccaaggcaacgactgcgccggccactacgcgcaacaccggccgcggcggcgaggagaagaagaaggagaaggagaaggaggagcaggaggagcgcgagaccaagacccccgagtgccccagccacacccagcccctgggcgtgttcctgttcccccccaagcccaaggacaccctgatgatcagccgcacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 53 - DNA, 885 bp, Artificial Sequence
  GLP-1-hyFc11
cacggcgaaggcaccttcaccagcgacgtgagcagctacctggaaggccaggctgccaaggagttcatcgcctggctggtgaaaggcagaggcgctcagccacaggctgagggaagcctcgccaaggcaacgactgcgccggccactacgcgcaacaccggccgcggcggcgaggagaagaagaaggagaaggagaaggaggagcaggaggagcgcgagaccaagacccccgagtgccccagccacacccagcccctgggcgtgttcctgttcccccccaagcccaaggacaccctgatgatcagccgcacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 54 - protein, 276 aa, Artificial Sequence
  GLP-1-hyFc5
HGEGTFTSDVSSYLEGQAAKEFIAWLVKGRGRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK